(19) United States
(12) Patent Application Publication
(10) Pub. No.: US 2013/0273625 A1
(43) Pub. Date: Oct. 17, 2013

(54) METHODS FOR INCREASING MICROBIAL PRODUCTION OF ISOPRENE, ISOPRENOIDS, AND ISOPRENOID PRECURSOR MOLECULES USING GLUCOSE AND ACETATE CO-METABOLISM

(71) Applicant: Danisco US Inc., Palo Alto, CA (US)

(72) Inventors: Gopal K. CHOTANI, Cupertino, CA (US); Alex T. NIELSEN, Kokkedal (DK); Dmitrii V. VAVILINE, Palo Alto, CA (US)

(21) Appl. No.: 13/646,556

(22) Filed: Oct. 5, 2012

Related U.S. Application Data
(60) Provisional application No. 61/544,959, filed on Oct. 7, 2011.

(51) Int. Cl. C12P 5/02

(52) U.S. Cl. CPC C12P 5/026
USPC 435/157; 435/167; 435/166

(57) ABSTRACT

Provided herein are methods for the increased production of intracellular acetyl-CoA, mevalonate, isoprenoid precursors, isoprene and/or isoprenoids by recombinant microorganisms via co-metabolism of substrates with varied oxidation levels.

This application claims priority to U.S. Provisional Patent Application No. 61/544,959, filed Oct. 7, 2011, the disclosure of which is incorporated by reference herein in its entirety.

### FIELD OF THE INVENTION

Compositions and methods for increasing the efficiency of the production of intracellular acetyl-CoA, mevalonate, isoprenoid precursors, isoprene and/or isoprenoids by recombinant microorganisms via co-metabolism of substrates with varied oxidation levels are described herein.

### BACKGROUND

R-Mevalonate is an intermediate of the mevalonate-dependent biosynthetic pathway that converts acetyl-CoA to isopentenyl diphosphate and dimethylallyl diphosphate. The conversion of acetyl-CoA to mevalonate can be catalyzed by the thiolase, HMG-CoA synthase and the HMG-CoA reductase activities of the upper mevalonate-dependent biosynthetic pathway (MVA pathway). Commercially, mevalonate has been used as an additive in cosmetics, for the production of biodegradable polymers, and can have value as a chiral building block for the synthesis of other chemicals. The lower mevalonate-dependent biosynthetic pathway utilizes mevalonate as substrate for generating isopentenyl pyrophosphate (IPP) and dimethylallyl diphosphate (DMAPP), which are the terminal products of the mevalonate-dependent pathway. IPP and DMAPP are precursors to isoprene as well as to isoprenoids.

Isoprene (2-methyl-1,3-butadiene) is an important organic compound used in a wide array of industrial applications. For example, isoprene is commonly employed as an intermediate or a starting material in the synthesis of many chemical compositions and polymers used in compositions to make synthetic rubber (natural rubber alternatives). Isoprene is also an important biological material that is synthesized naturally by many plants and animals.

Natural rubber supplies are limited and commercial production of isoprene from their natural sources raises environmental concerns. Commercially viable quantities of isoprene, instead, can be obtained by direct isolation from petroleum C5 cracking fractions or by dehydration of C5 isoalkanes or isoalkenes. The C5 skeleton can also be synthesized from smaller subunits.

Isoprenoids are compounds derived from the isoprenoid precursor molecules IPP and DMAPP. Over 29,000 isoprenoid compounds have been identified and new isoprenoids are being discovered each year. Isoprenoids can be isolated from natural products, such as microorganisms and species of plants that use isoprenoid precursor molecules as a basic building block to form the relatively complex structures of isoprenoids. Isoprenoids are vital to most living organisms and cells, providing a means to maintain cellular membrane fluidity and electron transport. In nature, isoprenoids function in roles as diverse as natural pesticides in plants to contributing to the scents associated with cinnamon, cloves, and ginger. Moreover, the pharmaceutical and chemical communities use isoprenoids as pharmaceuticals, nutraceuticals, flavoring agents, and agricultural pest control agents. Given their importance in biological systems and usefulness in a broad range of applications, isoprenoids have been the focus of much attention by scientists.

Bacterial production of isoprene also has been described (Kuzma et al., Curr Microbiol, 30: 97-103, 1995; and Wilkins, Chemosphere, 32: 1427-1434, 1996). Isoprene production varies in amount with the phase of bacterial growth and the nutrient content of the culture medium. See e.g., U.S. Pat. No. 5,849,970, U.S. Published Patent Application Nos. 2009/0203102, 2010/0003716, 2010/0086978, and Wagner et al., J Bacteriol, 181:4700-4703, 1999.

While several recent advancements have been made in the production of isoprene and related molecules by recombinant microorganisms, (See, for example, International Patent Application Publication No. WO 2010/148150 A2), process improvements to reduce the operational costs associated with production and to increase yields continue to be desired. What is needed, therefore, are improved methods for culturing isoprene, isoprenoid, and isoprenoid precursor-producing microorganisms using inexpensive carbon sources to optimize yield, efficiency, and productivity as well as to reduce the costs associated with production.

Throughout this specification, references are made to publications (e.g., scientific articles), patent applications, patents, etc., all of which are herein incorporated by reference in their entirety.

### BRIEF SUMMARY OF THE INVENTION

Disclosed herein are compositions and methods for increased production of intracellular acetyl-CoA concentrations, mevalonate, isoprenoid precursor molecules, isoprene and/or isoprenoids. In one aspect, the compositions and methods solve a problem that the microbial production of isoprene from glucose alone by the mevalonate (MVA) pathway results in a metabolic imbalance in the amount of reducing equivalents (for example, NADPH) produced by the microorganisms during metabolism. This imbalance limits the theoretical yield of isoprene and related molecules (e.g., mevalonate and/or isoprenoid precursors) which can be produced by the microorganisms in culture. However, co-metabolism of glucose and a more oxidized substrate, such as acetate, increases the yield and efficiency of production of mevalonate, isoprenoid precursors, isoprene, and isoprenoids by bringing the energy requirements of the cultured microorganisms more into balance. Additionally, the use of acetate as a carbon source further improves the production of these molecules, as it is converted by the cells into the important metabolic intermediate acetyl Co-A during the course of carbon metabolism.

The invention also provides further solutions to the problem of reducing CO₂ emissions during the production of mevalonate, isoprenoid precursors, isoprene, and/or isoprenoids.

Accordingly, provided herein are methods for improving the efficiency of the production of isoprene by recombinant host cells in culture, the methods comprising culturing said recombinant host cells in the presence of culture media comprising a carbon source and acetate under suitable conditions for the production of isoprene, wherein said recombinant host cells comprise one or more heterologous nucleic acids encoding an isoprene synthase polypeptide; wherein said recombinant host cells are capable of producing isoprene; and wherein isoprene production by said recombinant host cells cultured in the presence of culture media comprising a carbon source and acetate is improved compared to the isoprene production by said recombinant host cells cultured in the presence of culture media comprising a carbon source in the absence of acetate. In certain embodiments, the carbon source is glucose.

In other aspects, said improved efficiency of the production of isoprene is characterized by an increase in the specific productivity of isoprene. In one aspect, said increase in specific productivity of isoprene is at least about 10%. In another aspect, said increase in specific productivity of isoprene is at least about 20%. In another aspect, said increase in specific productivity of isoprene is at least about 30%. In another aspect, said increase in specific productivity of isoprene is at least about 40%. In another aspect, said increase in specific productivity of isoprene is at least about 50%. In another aspect, said increase in specific productivity of isoprene is at least about 60%. In another aspect, said increase in specific productivity of isoprene is at least about 70%. In another aspect, said increase in specific productivity of isoprene is at least about 80%. In another aspect, said increase in specific productivity of isoprene is at least about 90%. In another aspect, said increase in specific productivity of isoprene is at least about 100%.

In other aspects, said improved efficiency of the production of isoprene is characterized by an increase the cumulative yield of isoprene. In one aspect, said increase in cumulative yield of isoprene is at least about 1% to about 15%. In another aspect, said increase in cumulative yield of isoprene is at least about 1%. In another aspect, said increase in cumulative yield of isoprene is at least about 2%. In another aspect, said increase in cumulative yield of isoprene is at least about 3%. In another aspect, said increase in cumulative yield of isoprene is at least about 4%. In another aspect, said increase in cumulative yield of isoprene is at least about 5%. In another aspect, said increase in cumulative yield of isoprene is at least about 6%. In another aspect, said increase in cumulative yield of isoprene is at least about 7%. In another aspect, said increase in cumulative yield of isoprene is at least about 8%. In another aspect, said increase in cumulative yield of isoprene is at least about 9%. In another aspect, said increase in cumulative yield of isoprene is at least about 10%.

In other aspects, said improved efficiency of the production of isoprene is characterized by an increase in the cumulative yield over the preceding 40-hr period of isoprene. In one aspect, said increase in cumulative yield of isoprene over the preceding 40-hr period is at least about 1% to about 15%. In another aspect, said increase in cumulative yield of isoprene over the preceding 40-hr period is at least about 1%. In another aspect, said increase in cumulative yield of isoprene over the preceding 40-hr period is at least about 2%. In another aspect, said increase in cumulative yield of isoprene over the preceding 40-hr period is at least about 3%. In another aspect, said increase in cumulative yield of isoprene over the preceding 40-hr period is at least about 4%. In another aspect, said increase in cumulative yield of isoprene over the preceding 40-hr period is at least about 5%. In another aspect, said increase in cumulative yield of isoprene over the preceding 40-hr period is at least about 6%. In another aspect, said increase in cumulative yield of isoprene over the preceding 40-hr period is at least about 7%. In another aspect, said increase in cumulative yield of isoprene over the preceding 40-hr period is at least about 8%. In another aspect, said increase in cumulative yield of isoprene over the preceding 40-hr period is at least about 9%. In another aspect, said increase in cumulative yield of isoprene over the preceding 40-hr period is at least about 10%.

Cell Performance Index (CPI). In one aspect, said increase in CPI of isoprene is at least about 1% to about 15%. In another aspect, said increase in CPI of isoprene is at least about 1%. In another aspect, said increase in CPI of isoprene is at least about 2%. In another aspect, said increase in CPI of isoprene is at least about 3%. In another aspect, said increase in CPI of isoprene is at least about 4%. In another aspect, said increase in CPI of isoprene is at least about 5%. In another aspect, said increase in CPI of isoprene is at least about 6%. In another aspect, said increase in CPI of isoprene is at least about 7%. In another aspect, said increase in CPI of isoprene is at least about 8%. In another aspect, said increase in CPI of isoprene is at least about 9%. In another aspect, said increase in CPI of isoprene is at least about 10%.

In some aspects, said improved efficiency of the production of isoprene is characterized by an increase in the ratio between isoprene and carbon dioxide (CO₂). In another aspect, said increase in the ratio between isoprene and CO₂ is in fermentation off-gas. In one aspect, said increase in the ratio between isoprene and CO₂ is at least about 5%. In another aspect, said increase in the ratio between isoprene and CO₂ is at least about 10%. In another aspect, said increase in the ratio between isoprene and CO₂ is at least about 15%. In another aspect, said increase in the ratio between isoprene and CO₂ is at least about 20%. In another aspect, said increase in the ratio between isoprene and CO₂ is at least about 25%. In another aspect, said increase in the ratio between isoprene and CO₂ is at least about 30%. In another aspect, said increase in the ratio between isoprene and CO₂ is at least about 35%. In another aspect, said increase in the ratio between isoprene and CO₂ is at least about 40%. In another aspect, said increase in the ratio between isoprene and CO₂ is at least about 45%. In another aspect, said increase in the ratio between isoprene and CO₂ is at least about 50%. In another aspect, said increase in the ratio between isoprene and CO₂ is at least about 55%. In another aspect, said increase in the ratio between isoprene and CO₂ is at least about 60%.

In some aspects, the isoprene synthase polypeptide is a plant isoprene synthase polypeptide. In another aspect, the isoprene synthase polypeptide is a polypeptide from Pueraria or Populus or a hybrid, Populus alba×Populus tremula. In one aspect, the isoprene synthase polypeptide is selected from the group consisting of Pueraria montana, Pueraria lobata, Populus tremuloides, Populus alba, Populus nigra, and Populus trichocarpa. In another aspect, the plant isoprene synthase polypeptide is a kudzu isoprene synthase polypeptide.

In other aspects, the cells further comprise a heterologous nucleic acid encoding an isopentyl-diphosphate isomerase (IDI) polypeptide. In yet other aspects, the cells further comprise a chromosomal copy of an endogenous nucleic acid encoding an IDI polypeptide.

In other aspects, the cells further comprise one or more heterologous nucleic acid encoding one or more MVA pathway polypeptides. In another aspect, the cells further comprise one or more heterologous nucleic acid encoding two or more MVA pathway polypeptides. In another aspect, the cells further comprise one or more heterologous nucleic acid encoding three or more MVA pathway polypeptides. In another aspect, the cells further comprise one or more heterologous nucleic acid encoding four or more MVA pathway polypeptides. In yet another aspect, the cells further comprise one or more heterologous nucleic acid encoding the entire MVA pathway. In other aspect, the cells further comprise one or more heterologous nucleic acid encoding the upper MVA pathway. In some aspects, the cells further comprise one or more heterologous nucleic acids encoding MVA pathway polypeptides are from the lower MVA pathway. In other aspects, the lower MVA pathway nucleic acids are selected from the group consisting of MVK, PMK, and, MVD nucleic acids. In some aspects, the MVK is selected from the group consisting of Methanosarcina mazei mevalonate kinase, Methanococcoides burtonii mevalonate kinase polypeptide, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, and Streptomyces CL190 mevalonate kinase polypeptide.

In some aspects, the one or more heterologous nucleic acids are placed under an inducible promoter or a constitutive promoter. In some aspects, the one or more heterologous nucleic acids is cloned into a multicopy plasmid. In another aspect, the one or more heterologous nucleic acids is integrated into a chromosome of the cells.

In other aspects, the cells can further comprise a heterologous nucleic acid encoding a DXS polypeptide. In one aspect, the cells further comprise a chromosomal copy of an endogenous nucleic acid encoding a DXS polypeptide. In another aspect, the cells further comprise one or more nucleic acids encoding an IDI polypeptide, one or more MVA pathway polypeptides and/or a DXS polypeptide. In other aspects, one nucleic acid encodes the isoprene synthase polypeptide, IDI polypeptide, and DXS polypeptide. In another aspect, one plasmid encodes the isoprene synthase polypeptide, IDI polypeptide, and DXS polypeptide.

In some aspects, the one or more heterologous nucleic acids are placed under an inducible promoter or a constitutive promoter. In some aspects, the one or more heterologous nucleic acids is cloned into a multicopy plasmid. In another aspect, the one or more heterologous nucleic acids is integrated into a chromosome of the cells.

In some aspects, the recombinant host cells are bacterial cells. In another aspect, the recombinant host cells are gram-positive bacterial cells. In one aspect, the cells are Bacillus cells. In some aspects, the cells are Bacillus subtilis cells. In another aspect, the recombinant host cells are gram-negative bacterial cells. In yet another aspect, the cells are Escherichia or Pantoea cells. In one aspect, the cells are Escherichia coli or Pantoea citrea cells. In other aspects, the recombinant host cells are fungal cells. In one aspect, the cells are Trichoderma cells. In yet another aspect, the cells are Trichoderma reesei. In another aspect, the recombinant host cells are yeast cells. In another aspect, the yeast cells are selected from the group consisting of Saccharomyces sp., Schizosaccharomyces sp., Pichia sp., Candida sp, and Yarrowia sp. In another aspect, the yeast cells are Saccharomyces cerevisiae cells. In other aspects, the yeast cells are Yarrowia lipolytica cells.

In some aspects, the concentration of acetate is at least about 0.01% to about 1.5%. In one aspect, the concentration of acetate is at least about 0.01% to about 1.0%. In one aspect, the concentration of acetate is at least about 0.01% to about 0.75%. In one aspect, the concentration of acetate is at least about 0.01% to about 0.5%. In one aspect, the concentration of acetate is at least about 0.01% to about 0.4%. In one aspect, the concentration of acetate is at least about 0.01% to about 0.3%. In one aspect, the concentration of acetate is at least about 0.01% to about 0.25%. In one aspect, the concentration of acetate is at least about 0.01% to about 0.2%.

In some aspects, a method for improving the efficiency of the production of isoprenoid precursor molecules (e.g., mevalonate (MVA)) (e.g., mevalonate) by recombinant host cells in culture is provided, the method comprising culturing said recombinant host cells in the presence of culture media comprising a carbon source and acetate under suitable conditions for the production of isoprenoid precursor molecules (e.g., mevalonate (MVA)), wherein said recombinant host cells comprise one or more heterologous nucleic acids encoding one or more MVA pathway polypeptides; wherein said recombinant host cells are capable of producing isoprenoid precursor molecules; and wherein isoprenoid precursor molecule production by said recombinant host cells cultured in the presence of culture media comprising the carbon source and acetate is improved compared to the isoprenoid precursor molecule production by said recombinant host cells cultured in the presence of culture media comprising the carbon source in the absence of acetate. In certain embodiments, the carbon source is glucose.

In certain aspects, said improved efficiency of the production of isoprenoid precursors is characterized by an increase in the specific productivity. In one aspect, said increase in specific productivity of isoprenoid precursors is at least about 10% to about 100%. In another aspect, said increase in specific productivity of isoprenoid precursors is at least about 10%. In another aspect, said increase in specific productivity of isoprenoid precursors is at least about 20%. In another aspect, said increase in specific productivity of isoprenoid precursors is at least about 30%. In another aspect, said increase in specific productivity of isoprenoid precursors is at least about 40%. In another aspect, said increase in specific productivity of isoprenoid precursors is at least about 50%. In another aspect, said increase in specific productivity of isoprenoid precursors is at least about 60%. In another aspect, said increase in specific productivity of isoprenoid precursors is at least about 70%. In another aspect, said increase in specific productivity of isoprenoid precursors is at least about 80%. In another aspect, said increase in specific productivity of isoprenoid precursors is at least about 90%. In another aspect, said increase in specific productivity of isoprenoid precursors is at least about 100%.

In certain aspects, said improved efficiency of the production of isoprenoid precursors is characterized by an increase in isoprenoid precursor yield. In one aspect, said increase in yield of isoprenoid precursors is at least about 10% to about 100%. In another aspect, said increase in yield of isoprenoid precursors is at least about 10%. In another aspect, said increase in yield of isoprenoid precursors is at least about 20%. In another aspect, said increase in yield of isoprenoid precursors is at least about 30%. In another aspect, said increase in yield of isoprenoid precursors is at least about 40%. In another aspect, said increase in yield of isoprenoid precursors is at least about 50%. In another aspect, said increase in yield of isoprenoid precursors is at least about 60%. In another aspect, said increase in yield of isoprenoid precursors is at least about 70%. In another aspect, said increase in yield of isoprenoid precursors is at least about 80%. In another aspect, said increase in yield of isoprenoid precursors is at least about 90%. In another aspect, said increase in yield of isoprenoid precursors is at least about 100%.

In certain aspects, said improved efficiency of the production of isoprenoid precursors is characterized by an increase in the isoprenoid precursor titer. In one aspect, said increase in titer of isoprenoid precursors is at least about 5% to about 50%. In another aspect, said increase in titer of isoprenoid precursors is at least about 5%. In another aspect, said increase in titer of isoprenoid precursors is at least about 10%. In another aspect, said increase in titer of isoprenoid precursors is at least about 15%. In another aspect, said increase in titer of isoprenoid precursors is at least about 20%. In another aspect, said increase in titer of isoprenoid precursors is at least about 25%. In another aspect, said increase in titer of isoprenoid precursors is at least about 30%. In another aspect, said increase in titer of isoprenoid precursors is at least about 35%. In another aspect, said increase in titer of isoprenoid precursors is at least about 40%. In another aspect, said increase in titer of isoprenoid precursors is at least about 45%. In another aspect, said increase in titer of isoprenoid precursors is at least about 50%.

In certain embodiments, the isoprenoid precursor is selected from group consisting of mevalonate (MVA), DMAPP or IPP. In one embodiment, the isoprenoid precursor is mevalonate (MVA).

In certain embodiments, the carbon source is glucose. In one embodiment, the cells comprise one or more heterologous nucleic acid(s) encoding two or more MVA pathway polypeptides. In another embodiment, the cells comprise one or more heterologous nucleic acid(s) encoding three or more MVA pathway polypeptides. In another embodiment, the cells comprise one or more heterologous nucleic acid(s) encoding four or more MVA pathway polypeptides. In yet another embodiment, the cells comprise one or more heterologous nucleic acid encoding the entire MVA pathway. In certain aspects, the cells comprise one or more heterologous nucleic acid(s) encoding the upper MVA pathway. In some aspects, the cells further comprise one or more heterologous nucleic acids encoding MVA pathway polypeptides are from the lower MVA pathway. In other aspects, the lower MVA pathway nucleic acids are selected from the group consisting of MVK, PMK, and, MVD nucleic acids. In some aspects, the MVK is selected from the group consisting of Methanosarcina mazei mevalonate kinase, Methanococcoides burtonii mevalonate kinase polypeptide, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, and Streptomyces CL190 mevalonate kinase polypeptide.

In other aspects, the one or more heterologous nucleic acids is operatively linked to an inducible promoter or a constitutive promoter. In other aspects, the one or more heterologous nucleic acids is cloned into a multicopy plasmid.

In still another aspect, the one or more heterologous nucleic acids is integrated into a chromosome of the cells. In other aspects, the cells are selected from the group consisting of bacterial cells, fungal cells, or algal cells.

In some aspects, the concentration of acetate is at least about 0.01% to about 1.5%. In one aspect, the concentration of acetate is at least about 0.01% to about 1.0%. In another aspect, the concentration of acetate is at least about 0.01% to about 0.75%. In another aspect, the concentration of acetate is at least about 0.01% to about 0.5%. In another aspect, the concentration of acetate is at least about 0.01% to about 0.4%. In another aspect, the concentration of acetate is at least about 0.01% to about 0.3%. In another aspect, wherein the concentration of acetate is at least about 0.01% to about 0.25%. In another aspect, wherein the concentration of acetate is at least about 0.01% to about 0.2%.

In some aspects, a method for improving the efficiency of the production of isoprenoids by recombinant host cells in culture is provided, the method comprising culturing said recombinant host cells in the presence of culture media comprising a carbon source and acetate under suitable conditions for the production of isoprenoids, wherein said recombinant host cells comprise (i) one or more heterologous nucleic acids encoding one or more MVA pathway polypeptides and (ii) one or more heterologous nucleic acids encoding a polyprenyl pyrophosphate synthase; wherein said recombinant host cells are capable of producing isoprenoids; and wherein isoprenoid production by said recombinant host cells cultured in the presence of culture media comprising the carbon source and acetate is improved compared to the isoprenoid production by said recombinant host cells cultured in the presence culture media comprising the carbon source in the absence of acetate. In certain embodiments, the carbon source is glucose.

In one embodiment, the cells comprise one or more heterologous nucleic acid encoding two or more MVA pathway polypeptides. In another embodiment, the cells comprise one or more heterologous nucleic acid encoding three or more MVA pathway polypeptides. In another embodiment, the cells comprise one or more heterologous nucleic acid encoding four or more MVA pathway polypeptides. In yet another embodiment, the cells comprise one or more heterologous nucleic acid encoding the entire MVA pathway. In certain aspects, the cells comprise one or more heterologous nucleic acid encoding the upper MVA pathway. In some aspects, the cells further comprise one or more heterologous nucleic acids encoding MVA pathway polypeptides are from the lower MVA pathway. In other aspects, the lower MVA pathway nucleic acids are selected from the group consisting of MVK, PMK, and, MVD nucleic acids. In some aspects, the MVK is selected from the group consisting of Methanosarcina mazei mevalonate kinase, Methanococcoides burtonii mevalonate kinase polypeptide, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, and Streptomyces CL190 mevalonate kinase polypeptide.

In some aspects, said improved efficiency of the production of an isoprenoid is characterized by an increase in the specific productivity of said isoprenoid.

In other aspect, the one or more heterologous nucleic acids is operatively linked to an inducible promoter or a constitutive promoter. In other aspects, the one or more heterologous nucleic acids is cloned into a multicopy plasmid.

In still another aspect, the one or more heterologous nucleic acids is integrated into a chromosome of the cells. In other aspects, the cells are selected from the group consisting of bacterial cells, fungal cells, or algal cells.

In other aspects, the isoprenoid is selected from group consisting of monoterpenes, diterpenes, triterpenes, tetraterpenes, sequiterpene, and polyterpene. In one aspect, the isoprenoid is a sesquiterpene. In other aspects, the isoprenoid is selected from the group consisting of abietadiene, amorphadiene, carene, α-farnesene, β-farnesene, farnesol, geraniol, geranylgeraniol, linalool, limonene, myrcene, nerolidol, ocimene, patchoulol, β-pinene, sabinene, γ-terpinene, terpindene and valencene.

In some aspects, the concentration of acetate is at least about 0.01% to about 1.5%. In one aspect, the concentration of acetate is at least about 0.01% to about 1.0%. In another aspect, the concentration of acetate is at least about 0.01% to about 0.75%. In another aspect, the concentration of acetate is at least about 0.01% to about 0.5%. In another aspect, the concentration of acetate is at least about 0.01% to about 0.4%. In another aspect, the concentration of acetate is at least about 0.01% to about 0.3%. In another aspect, wherein the concentration of acetate is at least about 0.01% to about 0.25%. In another aspect, wherein the concentration of acetate is at least about 0.01% to about 0.2%.

In some aspects, a method for increasing the intracellular concentration of acetyl Co-A in transformed host cells is provided, the method comprising culturing said transformed host cells in the presence of culture media comprising a carbon source and acetate under suitable conditions for the production of intracellular acetyl Co-A, wherein said transformed host cells are capable of producing intracellular acetyl Co-A; and wherein the intracellular acetyl Co-A in said transformed host cells cultured in the presence of culture media comprising a carbon source and acetate is increased compared to the intracellular acetyl Co-A in said transformed host cells cultured in the presence of culture media comprising a carbon source in the absence of acetate. In certain embodiments, the carbon source is glucose.

In some aspects, a method for reducing carbon dioxide emissions in the production of isoprene by recombinant host cells in culture is provided, the method comprising culturing said recombinant host cells in the presence of culture media comprising a carbon source and acetate under suitable conditions for the production of isoprene, wherein said recombinant host cells comprise one or more heterologous nucleic acids encoding an isoprene synthase polypeptide; wherein said recombinant host cells are capable of producing isoprene; and wherein isoprene production by said recombinant host cells cultured in the presence of culture media comprising a carbon source and acetate is improved compared to the isoprene production by said recombinant host cells cultured in the presence of culture media comprising a carbon source in the absence of acetate. In certain embodiments, the carbon source is glucose.

In some aspects, a method for reducing carbon dioxide emissions in the production of isoprenoids and/or isoprenoid precursor molecules (e.g., mevalonate (MVA)) by a recombinant host cell is provided, the method comprising culturing said recombinant host cells in the presence of glucose and acetate under suitable conditions for the production of isoprenoids and/or isoprenoid precursor molecules, wherein said recombinant host cells comprise one or more heterologous nucleic acids encoding a polyprenyl pyrophosphate synthase; wherein said recombinant host cells are capable of producing isoprenoids and/or isoprenoid precursor molecules; and wherein isoprenoid and/or isoprenoid precursor molecule production by said recombinant host cells cultured in the presence of glucose and acetate is improved compared to the isoprenoid and/or isoprenoid precursor molecule production by said recombinant host cells cultured in the presence of glucose alone.

### BRIEF DESCRIPTION OF THE DRAWINGS

FIG. 1 depicts a schematic representation for co-culturing homoacetogenic microorganisms in parallel with recombinant microorganisms engineered for the production of isoprene, isoprenoid, and/or isoprenoid precursor molecules. The system utilizes an acetate storage tank.

FIG. 2 depicts a schematic representation for co-culturing homoacetogenic microorganisms in parallel with recombinant microorganisms engineered for the production of isoprene, isoprenoid, and/or isoprenoid precursor molecules. The system utilizes direct acetate feeding mechanism.

FIG. 3 depicts a schematic representation for co-culturing homoacetogenic microorganisms in parallel with recombinant microorganisms engineered for the production of isoprene, isoprenoid, and/or isoprenoid precursor molecules. The system utilizes an oxygen gradient in the isoprene, isoprenoid, and/or isoprenoid precursor molecule fermentor.

FIG. 4 depicts respiration during addition of acetate to a glucose batch culture of E. coli.

FIG. 5 depicts isoprene production measured as μg/L offgas during addition of acetate to a glucose batch culture of E. coli.

FIG. 6 depicts the ratio between the percentage of isoprene and percentage of CO₂ produced during acetate and glucose co-metabolism by E. coli.

FIG. 7 depicts the ratio between the percentage of isoprene and percentage of CO₂ produced during acetate and glucose co-metabolism by E. coli observed at two induction levels of the MVA pathway. (A) 100 μM IPTG induction, (B) 200 μM IPTG induction.

FIG. 8 depicts distribution of ¹³C/¹²C in the acetyl group of acetyl-Co-A during growth at different levels of ¹³C-labeled glucose and ¹²C-labeled acetate.

FIG. 9 depicts the effect of acetate and glucose co-metabolism on the specific isoprene productivity and the intracellular concentration of acetyl-Co-A.

FIG. 10 depicts the effect of acetate on MVA specific productivity in CHL936 strain. MVA specific productivity was calculated as the amount of MVA produced during 3.5 h incubation period after adding specified amounts of sodium acetate to the cells and normalized to OD600 reached by the cultures at the end of the 3.5 hr incubation period. Shown are the average data ±SD of two independent measurements.

FIG. 11 depicts the effect of acetate on MVA yield on glucose in CHL936 strain. MVA yield on glucose was calculated as the amount of MVA produced divided by the amount of glucose consumed during 3.5 h incubation period after adding specified amounts of sodium acetate to the cells. Shown are the average results of two independent measurements.

FIG. 12 depicts the dynamics of cell growth in a 15-L fermentor in acetate+glucose fed culture of DW719 strain as compared to the control growing without the acetate co-feed. The acetate was fed during the period indicated by grey bar at the bottom of the chart.

FIG. 13 depicts accumulation of acetate in fermentor broth in glucose+acetate-fed culture of DW719 strain as compared to the control grown on glucose without the acetate co-feed.

FIG. 14 depicts cumulative yield of isoprene on glucose in glucose+acetate-fed culture of DW719 strain as compared to the control grown on glucose without the acetate co-feed.

FIG. 15 depicts cumulative isoprene yield over the preceding 40-hr period in glucose+acetate-fed culture of DW719 strain as compared to the control grown on glucose without the acetate co-feed.

FIG. 16 depicts Cell Productivity Index (CPI) over the preceding 40-hr period in glucose+acetate-fed culture of DW719 strain as compared to the control grown on glucose without the acetate co-feed

### DETAILED DESCRIPTION

The invention provides, inter alia, compositions and methods for the increased production of isoprene, isoprenoids, isoprenoid precursor molecules, and intracellular acetyl Co-A concentrations by recombinant microorganisms by culturing those microorganisms in the presence of substrates with varied oxidation states. By balancing the reducing equivalents of the various reactions during production of isoprene, isoprenoids, and isoprenoid precursor molecules, the yield and/or productivity of the products can be increased.

Accordingly, in one aspect, the invention provides for compositions and methods for the production of isoprene, isoprenoids, and/or isoprenoid precursor molecules (e.g., mevalonate (MVA)) by culturing recombinant microorganisms engineered for increased carbon flux towards the mevalonate (MVA) biosynthetic pathway in the presence of glucose and acetate under suitable conditions for the production of these molecules. In other aspects, the invention provides methods for increasing intracellular concentrations of acetyl Co-A in recombinant microorganisms by culturing those recombinant microorganisms in the presence of glucose and acetate. In an additional aspect, the invention provides methods for decreasing carbon dioxide emissions during the production of isoprene, isoprenoids, and/or isoprenoid precursor molecules by culturing recombinant microorganisms engineered for the production of these molecules in the presence of glucose and acetate.

Production of isoprene via the mevalonate (MVA) pathway in recombinant microorganisms grown on glucose alone results in a theoretical yield of 25.2% and an imbalance in the amount of reducing equivalents produced in the form of NAD(P)H (or ATP). This imbalance in reducing equivalents may affect the yield of isoprene actually obtained from the culture. Without being bound to theory, it is believed that co-metabolism of glucose and a more oxidized substrate, such as acetate, can increase yields of mevalonate, isoprenoid precursor molecules, isoprene, and isoprenoids via more efficient balancing of the cells' energy requirements. The equations below demonstrate how co-metabolism of glucose and acetate can theoretically increase the mass yield of isoprene produced in culture. The equations also demonstrate how co-metabolism of glucose and acetate can decrease the amount of oxygen required as well as the amount of CO₂ emitted by the production process.

### Equations 1-3: Glucose Metabolism Alone:

1½C₆H₁₂O₆+2O₂→C₅H₈+4CO₂+(12ATP); (1)

1½C₆H₁₂O₆→C₅H₈+4CO₂+4 NAD(P)H or (2)

1½C₆H₁₂O₆+2O₂→C₅H₈+4CO₂+5 H2O (3)

Maximum theoretical mass yield: 25.2%

As is evident from the result of Equations 1-3, the excess ATP (or NADH) produced from the metabolism of glucose alone in the isoprene production process will need to be consumed, thus resulting in a lower theoretical yield.

### Equation 4: Glucose and Acetate Co-Metabolism:

5/6 C₆H₁₂O₆+4/3 CH₃COOH+5/3 O₂→C₅H₈+22/3 CO₂ (4)

Maximum theoretical mass yield: 29.6%

As seen in Equation 4, however, co-metabolism of glucose and acetate can result in minimal excess (e.g. no excess) production of reducing equivalents as well as a reaction that can require less overall oxygen and results in less CO₂ production than the reaction shown in Equation 1.

Additionally, as detailed herein, the mevalonate-dependent biosynthetic pathway is particularly important for the production of the isoprenoid precursor molecules dimethylallyl diphosphate (DMAPP) and isopentenyl pyrophosphate (IPP). The enzymes of the upper mevalonate pathway convert acetyl Co-A, derived from metabolic substrates such as glucose and acetate, into mevalonate which then is converted to DMAPP and IPP via the enzymes of the lower MVA pathway. Therefore, increasing carbon flux in microorganisms engineered for the production of isoprene, isoprenoids, and isoprenoid precursor molecules towards the MVA pathway can lead to an increase in the overall production of these molecules.

Without being bound to theory, it is believed that culturing these recombinant microorganisms in the presence of acetate can increase the concentration of acetyl Co-A in the cell due to the fact that acetate is converted into acetyl Co-A during cellular metabolism. Therefore, culturing recombinant cells engineered for the production of isoprene, isoprenoids, and isoprenoid precursor molecules (e.g., mevalonate (MVA)) in the presence of both glucose and acetate can further increase carbon flux through the MVA pathway due to increased intracellular acetyl Co-A concentrations, thereby resulting in increased production of these molecules.

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994). Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

### DEFINITIONS

The term “isoprene” refers to 2-methyl-1,3-butadiene (CAS#78-79-5). It can be the direct and final volatile C5 hydrocarbon product from the elimination of pyrophosphate from 3,3-dimethylallyl diphosphate (DMAPP). It may not involve the linking or polymerization of IPP molecules to DMAPP molecules. The term “isoprene” is not generally intended to be limited to its method of production unless indicated otherwise herein.

As used herein, the term “polypeptides” includes polypeptides, proteins, peptides, fragments of polypeptides, and fusion polypeptides.

As used herein, an “isolated polypeptide” is not part of a library of polypeptides, such as a library of 2, 5, 10, 20, 50 or more different polypeptides and is separated from at least one component with which it occurs in nature. An isolated polypeptide can be obtained, for example, by expression of a recombinant nucleic acid encoding the polypeptide.

By “heterologous polypeptide” is meant a polypeptide encoded by a nucleic acid sequence derived from a different organism, species, or strain than the host cell. In some aspects, a heterologous polypeptide is not identical to a wild-type polypeptide that is found in the same host cell in nature.

As used herein, a “nucleic acid” refers to two or more deoxyribonucleotides and/or ribonucleotides covalently joined together in either single or double-stranded form.

By “recombinant nucleic acid” is meant a nucleic acid of interest that is free of one or more nucleic acids (e.g., genes) which, in the genome occurring in nature of the organism from which the nucleic acid of interest is derived, flank the nucleic acid of interest. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA, a genomic DNA fragment, or a cDNA fragment produced by PCR or restriction endonuclease digestion) independent of other sequences.

By “heterologous nucleic acid” is meant a nucleic acid sequence derived from a different organism, species or strain than the host cell. In some aspects, the heterologous nucleic acid is not identical to a wild-type nucleic acid that is found in the same host cell in nature.

As used herein, an “expression control sequence” means a nucleic acid sequence that directs transcription of a nucleic acid of interest. An expression control sequence can be a promoter, such as a constitutive or an inducible promoter, or an enhancer. An expression control sequence can be “native” or heterologous. A native expression control sequence is derived from the same organism, species, or strain as the gene being expressed. A heterologous expression control sequence is derived from a different organism, species, or strain as the gene being expressed. An “inducible promoter” is a promoter that is active under environmental or developmental regulation.

By “operably linked” is meant a functional linkage between a nucleic acid expression control sequence (such as a promoter) and a second nucleic acid sequence, wherein the expression control sequence directs transcription of the nucleic acid corresponding to the second sequence.

As used herein, the terms “minimal medium” or “minimal media” refer to growth medium containing the minimum nutrients possible for cell growth, generally without the presence of amino acids. Minimal medium typically contains: (1) a carbon source for bacterial growth; (2) various salts, which can vary among bacterial species and growing conditions; and (3) water. The carbon source can vary significantly, from simple sugars like glucose to more complex hydrolysates of other biomass, such as yeast extract, as discussed in more detail below. The salts generally provide essential elements such as magnesium, nitrogen, phosphorus, and sulfur to allow the cells to synthesize proteins and nucleic acids. Minimal medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

As used herein, the term “isoprenoid” refers to a large and diverse class of naturally-occurring class of organic compounds composed of two or more units of hydrocarbons, with each unit consisting of five carbon atoms arranged in a specific pattern. As used herein, “isoprene” is expressly excluded from the definition of “isoprenoid.”

As used herein, the term “terpenoid” refers to a large and diverse class of organic molecules derived from five-carbon isoprenoid units assembled and modified in a variety of ways and classified in groups based on the number of isoprenoid units used in group members. Monoterpenoids have two isoprenoid units. Sesquiterpenoids have three isoprenoid units. Diterpenoids have four isoprene units. Sesterterpenoids have five isoprenoid units. Triterpenoids have six isoprenoid units. Tetraterpenoids have eight isoprenoid units. Polyterpenoids have more than eight isoprenoid units.

As used herein, “isoprenoid precursor” refers to any molecule that is used by organisms in the biosynthesis of terpenoids or isoprenoids. Non-limiting examples of isoprenoid precursor molecules include, e.g., mevalonate, isopentenyl pyrophosphate (IPP) and dimethylallyl diphosphate (DMAPP).

As used herein, the term “mass yield” refers to the mass of the product produced by the recombinant cells (e.g., bacterial cells) divided by the mass of the glucose consumed by the bacterial cells multiplied by 100.

By “specific productivity,” it is meant the mass of the product produced by the recombinant cells (e.g., bacterial cells) divided by the product of the time for production, the cell density, and the volume of the culture.

By “titer,” it is meant the mass of the product produced by the recombinant cells (e.g., bacterial cells) divided by the volume of the culture.

As used herein, the term “cell productivity index (CPI)” refers to the mass of the product produced by the recombinant cells (e.g., bacterial cells) divided by the mass of the bacterial cells produced in the culture.

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Engineering Recombinant Cells Capable of Isoprene Production

Microorganisms can be engineered to produce isoprene. The cells can be engineered to contain a heterologous nucleic acid encoding an isoprene synthase polypeptide. The cells can be further engineered to include nucleic acids for one or more MVA pathway polypeptide or DXP pathway polypeptides or nucleic acids for both pathways. Various isoprene synthase polypeptides, DXP pathway polypeptides, IDI polypeptides, MVA pathway polypeptides, and nucleic acids can be used in the methods disclosed herein. Exemplary nucleic acids, polypeptides and enzymes that can be used are described in, for example, WO 2009/076676 and WO 2010/003007, both of which also include the Appendices listing exemplary nucleic acids and polypeptides for isoprene synthase, MVA pathway, acetyl-Co-A-acetyltransferase, HMG-Co-A synthase, hydroxymethylglutaryl-Co-A reductase, mevalonate kinase, phosphomevalonate kinase, diphosphomevalonate decarboxylase, isopentenyl phosphate kinases (IPK), DXP pathway, isopentenyl-diphosphate delta-isomerase (IDI) and other polypeptide and nucleic acids that one of skill in the art can use to make cells which produce isoprene.

Isoprene Synthase Polypeptides and Nucleic Acids

Exemplary isoprene synthase nucleic acids include nucleic acids that encode a polypeptide, fragment of a polypeptide, peptide, or fusion polypeptide that has at least one activity of an isoprene synthase polypeptide. Isoprene synthase polypeptides convert dimethylallyl diphosphate (DMAPP) into isoprene. Exemplary isoprene synthase polypeptides include polypeptides, fragments of polypeptides, peptides, and fusions polypeptides that have at least one activity of an isoprene synthase polypeptide. Exemplary isoprene synthase polypeptides and nucleic acids include naturally-occurring polypeptides and nucleic acids from any of the source organisms described herein. In addition, variants of isoprene synthase can possess improved activity such as improved enzymatic activity. In some aspects, an isoprene synthase variant has other improved properties, such as improved stability (e.g., thermo-stability), and/or improved solubility.

Standard methods can be used to determine whether a polypeptide has isoprene synthase polypeptide activity by measuring the ability of the polypeptide to convert DMAPP into isoprene in vitro, in a cell extract, or in vivo. Isoprene synthase polypeptide activity in the cell extract can be measured, for example, as described in Silver et al., J. Biol. Chem. 270:13010-13016, 1995. In one exemplary assay, DMAPP (Sigma) can be evaporated to dryness under a stream of nitrogen and rehydrated to a concentration of 100 mM in 100 mM potassium phosphate buffer pH 8.2 and stored at −200 C. To perform the assay, a solution of 5 μL of 1M MgCl₂, 1 mM (250 μg/ml) DMAPP, 65 μL of Plant Extract Buffer (PEB) (50 mM Tris-HCl, pH 8.0, 20 mM MgCl₂, 5% glycerol, and 2 mM DTT) can be added to 25 μL of cell extract in a 20 ml Headspace vial with a metal screw cap and teflon coated silicon septum (Agilent Technologies) and cultured at 370 C for 15 minutes with shaking. The reaction can be quenched by adding 200 μL of 250 mM EDTA and quantified by GC/MS.

In some aspects, the isoprene synthase polypeptide is a plant isoprene synthase polypeptide or a variant thereof. In some aspects, the isoprene synthase polypeptide is an isoprene synthase from Pueraria or a variant thereof. In some aspects, the isoprene synthase polypeptide is an isoprene synthase from Populus or a variant thereof. In some aspects, the isoprene synthase polypeptide is a poplar isoprene synthase polypeptide or a variant thereof. In some aspects, the isoprene synthase polypeptide is a kudzu isoprene synthase polypeptide or a variant thereof. In some aspects, the isoprene synthase polypeptide is a polypeptide from Pueraria or Populus or a hybrid, Populus alba×Populus tremula, or a variant thereof.

In some aspects, the isoprene synthase polypeptide or nucleic acid is from the family Fabaceae, such as the Faboideae subfamily. In some aspects, the isoprene synthase polypeptide or nucleic acid is a polypeptide or nucleic acid from Pueraria montana (kudzu) (Sharkey et al., Plant Physiology 137: 700-712, 2005), Pueraria lobata, poplar (such as Populus alba, Populus nigra, Populus trichocarpa, or Populus alba×tremula (CAC35696) (Miller et al., Planta 213: 483-487, 2001), aspen (such as Populus tremuloides) (Silver et al., JBC 270(22): 13010-1316, 1995), English Oak (Quercus robur) (Zimmer et al., WO 98/02550), or a variant thereof. In some aspects, the isoprene synthase polypeptide is an isoprene synthase from Pueraria montana, Pueraria lobata, Populus tremuloides, Populus alba, Populus nigra, or Populus trichocarpa or a variant thereof. In some aspects, the isoprene synthase polypeptide is an isoprene synthase from Populus alba or a variant thereof. In some aspects, the nucleic acid encoding the isoprene synthase (e.g., isoprene synthase from Populus alba or a variant thereof) is codon optimized.

In some aspects, the isoprene synthase nucleic acid or polypeptide is a naturally-occurring polypeptide or nucleic acid (e.g., naturally-occurring polypeptide or nucleic acid from Populus). In some aspects, the isoprene synthase nucleic acid or polypeptide is not a wild-type or naturally-occurring polypeptide or nucleic acid. In some aspects, the isoprene synthase nucleic acid or polypeptide is a variant of a wild-type or naturally-occurring polypeptide or nucleic acid (e.g., a variant of a wild-type or naturally-occurring polypeptide or nucleic acid from Populus).

In some aspects, the isoprene synthase polypeptide is a variant. In some aspects, the isoprene synthase polypeptide is a variant of a wild-type or naturally occurring isoprene synthase. In some aspects, the variant has improved activity such as improved catalytic activity compared to the wild-type or naturally occurring isoprene synthase. The increase in activity (e.g., catalytic activity) can be at least about any of 10%, 20%, 30%, 40%, 50%, 60%, 70%, 80%, 90%, or 95%. In some aspects, the increase in activity such as catalytic activity is at least about any of 1 fold, 2 folds, 5 folds, 10 folds, 20 folds, 30 folds, 40 folds, 50 folds, 75 folds, or 100 folds. In some aspects, the increase in activity such as catalytic activity is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the variant has improved solubility compared to the wild-type or naturally occurring isoprene synthase. The increase in solubility can be at least about any of 10%, 20%, 30%, 40%, 50%, 60%, 70%, 80%, 90%, or 95%. The increase in solubility can be at least about any of 1 fold, 2 folds, 5 folds, 10 folds, 20 folds, 30 folds, 40 folds, 50 folds, 75 folds, or 100 folds. In some aspects, the increase in solubility is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the isoprene synthase polypeptide is a variant of naturally occurring isoprene synthase and has improved stability (such as thermo-stability) compared to the naturally occurring isoprene synthase.

In some aspects, the variant has at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200% of the activity of a wild-type or naturally occurring isoprene synthase. The variant can share sequence similarity with a wild-type or naturally occurring isoprene synthase. In some aspects, a variant of a wild-type or naturally occurring isoprene synthase can have at least about any of 40%, 50%, 60%, 70%, 75%, 80%, 90%, 91%, 92%, 93%, 94%, 95%, 96%, 97%, 98%, 99%, 99.5%, or 99.9% amino acid sequence identity as that of the wild-type or naturally occurring isoprene synthase. In some aspects, a variant of a wild-type or naturally occurring isoprene synthase has any of about 70% to about 99.9%, about 75% to about 99%, about 80% to about 98%, about 85% to about 97%, or about 90% to about 95% amino acid sequence identity as that of the wild-type or naturally occurring isoprene synthase.

In some aspects, the variant comprises a mutation in the wild-type or naturally occurring isoprene synthase. In some aspects, the variant has at least one amino acid substitution, at least one amino acid insertion, and/or at least one amino acid deletion. In some aspects, the variant has at least one amino acid substitution. In some aspects, the number of differing amino acid residues between the variant and wild-type or naturally occurring isoprene synthase can be one or more, e.g. 1, 2, 3, 4, 5, 10, 15, 20, 30, 40, 50, or more amino acid residues. Naturally occurring isoprene synthases can include any isoprene synthases from plants, for example, kudzu isoprene synthases, poplar isoprene synthases, English oak isoprene synthases, and willow isoprene synthases. In some aspects, the variant is a variant of isoprene synthase from Populus alba. In some aspects, the variant of isoprene synthase from Populus alba has at least one amino acid substitution, at least one amino acid insertion, and/or at least one amino acid deletion. In some aspects, the variant is a truncated Populus alba isoprene synthase. In some aspects, the nucleic acid encoding variant (e.g., variant of isoprene synthase from Populus alba) is codon optimized (for example, codon optimized based on host cells where the heterologous isoprene synthase is expressed).

The isoprene synthase polypeptide provided herein can be any of the isoprene synthases or isoprene synthase variants described in WO 2009/132220, WO 2010/124146, and WO 2012/058494, the contents of which are expressly incorporated herein by reference in their entirety with respect to the isoprene synthases and isoprene synthase variants. In addition, types of isoprene synthases which can be used and methods of making microorganisms encoding isoprene synthase are also described in International Patent Application Publication No. WO 2009/076676; and U.S. Patent Application Publication Nos. 2010/0048964, 2010/0086978, 2010/0167370, 20100113846, 2010/0184178, 2010/0167371, 2010/0196977, 2011/0014672, and 2011/0046422, the contents of which are expressly incorporated herein by reference in their entirety. Additional suitable isoprene synthases include, but are not limited to, those identified by Genbank Accession Nos. AY341431, AY316691, AY279379, AJ457070, and AY182241.

In some aspects, the heterologous nucleic acid encoding any of the isoprene synthase polypeptides described herein can be expressed in the isoprene-producing cell on a multicopy plasmid. In other aspects, the nucleic acid encoding any of the isoprene synthase polypeptides described herein can be integrated into the chromosome of the host cell. In some aspects, the isoprene synthase nucleic acid is operably linked to a constitutive promoter or can alternatively be operably linked to an inducible promoter.

MVA Pathway Polypeptides and Nucleic Acids

In some aspects of the invention, the cells described in any of the methods described herein comprise one or more nucleic acid(s) encoding an MVA pathway polypeptide. In some aspects, the MVA pathway polypeptide is an endogenous polypeptide. In a particular aspect, the cells are engineered to over-express the endogenous MVA pathway polypeptide relative to wild-type cells. In some aspects, the cells comprise one or more additional copies of an endogenous nucleic acid encoding an MVA pathway polypeptide. In some aspects, the endogenous nucleic acid encoding an MVA pathway polypeptide is operably linked to a constitutive promoter. In some aspects, the endogenous nucleic acid encoding an MVA pathway polypeptide is operably linked to an inducible promoter. In another aspect, the MVA pathway polypeptide is a heterologous polypeptide. In some aspects, the heterologous nucleic acid encoding an MVA pathway polypeptide is operably linked to a constitutive promoter. In some aspects, the heterologous nucleic acid encoding an MVA pathway polypeptide is operably linked to an inducible promoter. In a particular aspect, the cells are engineered to over-express the heterologous MVA pathway polypeptide relative to wild-type cells.

Exemplary MVA pathway polypeptides include acetyl-Co-A acetyltransferase (AA-Co-A thiolase) polypeptides, acetoacetyl-CoA synthase polypeptides (which utilizes acetyl-CoA and malonyl-CoA as substrates (a.k.a., nphT7)), 3-hydroxy-3-methylglutaryl-Co-A synthase (HMG-Co-A synthase) polypeptides, 3-hydroxy-3-methylglutaryl-Co-A reductase (HMG-Co-A reductase) polypeptides, mevalonate kinase (MVK) polypeptides, phosphomevalonate kinase (PMK) polypeptides, diphosphomevalonate decarboxylase (MVD) polypeptides, phosphomevalonate decarboxylase (PMDC) polypeptides, isopentenyl phosphate kinase (IPK) polypeptides, IDI polypeptides, and polypeptides (e.g., fusion polypeptides) having an activity of two or more MVA pathway polypeptides. In particular, MVA pathway polypeptides include polypeptides, fragments of polypeptides, peptides, and fusions polypeptides that have at least one activity of an MVA pathway polypeptide. Exemplary MVA pathway nucleic acids include nucleic acids that encode a polypeptide, fragment of a polypeptide, peptide, or fusion polypeptide that has at least one activity of an MVA pathway polypeptide. Exemplary MVA pathway polypeptides and nucleic acids include naturally-occurring polypeptides and nucleic acids from any of the source organisms described herein. In addition, variants of MVA pathway polypeptide that confer the result of better isoprene production can also be used as well. In some aspects, the MVA pathway polypeptides can include the polypeptides encoded by any of the mvaE and mvaS genes of L. grayi, E. faecium, E. gallinarum, E. faecalis, and E. casseliflavus.

In some aspects, feedback resistant mevalonate kinase polypeptides can be used to increase the production of isoprene. As such, the invention provides methods for producing isoprene wherein the host cells further comprise (i) one or more non-modified nucleic acids encoding feedback-resistant mevalonate kinase polypeptides or (ii) one or more additional copies of an endogenous nucleic acid encoding a feedback-resistant mevalonate kinase polypeptide. Non-limiting examples of mevalonate kinase which can be used include: archaeal mevalonate kinase (e.g., from Methanosarcina (such as from M. mazei) or Methanococcoides (such as M. burtonii), Lactobacillus mevalonate kinase polypeptide, Lactobacillus sakei mevalonate kinase polypeptide, yeast mevalonate kinase polypeptide, Streptococcus mevalonate kinase polypeptide, Streptococcus pneumoniae mevalonate kinase polypeptide, Streptomyces mevalonate kinase polypeptide, and Streptomyces CL190 mevalonate kinase polypeptide.

Types of MVA pathway polypeptides which can be used and methods of making microorganisms (e.g., facultative anaerobes such as E. coli) encoding MVA pathway polypeptides are also described in International Patent Application Publication No. WO2009/076676; and U.S. Patent Application Publication Nos. 2010/0048964, 2010/0086978, 2010/0167370, 2010/0113846, 2010/0184178, 2010/0167371, 2010/0196977, 2011/0014672, and 2011/0046422.

In another aspect, aerobes are engineered with isoprene synthase using standard techniques known to one of skill in the art. In another aspect, anaerobes are engineered with isoprene synthase and one or more MVA pathway polypeptides and/or one or more DXP pathway polypeptides using standard techniques known to one of skill in the art. In yet another aspect, either aerobes or anaerobes are engineered with isoprene synthase, one or more MVA pathway polypeptides and/or one or more DXP pathway polypeptides using standard techniques known to one of skill in the art.

IDI Polypeptides and Nucleic Acids

Isopentenyl diphosphate isomerase polypeptides (isopentenyl-diphosphate delta-isomerase or IDI) catalyses the interconversion of isopentenyl diphosphate (IPP) and dimethyl allyl diphosphate (DMAPP) (e.g., converting IPP into DMAPP and/or converting DMAPP into IPP). While not intending to be bound by any particular theory, it is believed that increasing the amount of IDI polypeptide in cells increases the amount (and conversion rate) of IPP that is converted into DMAPP, which in turn is converted into isoprene. Exemplary IDI polypeptides include polypeptides, fragments of polypeptides, peptides, and fusions polypeptides that have at least one activity of an IDI polypeptide. Standard methods can be used to determine whether a polypeptide has IDI polypeptide activity by measuring the ability of the polypeptide to interconvert IPP and DMAPP in vitro, in a cell extract, or in vivo. Exemplary IDI nucleic acids include nucleic acids that encode a polypeptide, fragment of a polypeptide, peptide, or fusion polypeptide that has at least one activity of an IDI polypeptide. Exemplary IDI polypeptides and nucleic acids include naturally-occurring polypeptides and nucleic acids from any of the source organisms described herein as well as mutant polypeptides and nucleic acids derived from any of the source organisms described herein.

In some aspects, the heterologous nucleic acid encoding one or more of any of the IDI polypeptides described herein can be expressed in the isoprene-producing cell on a multicopy plasmid. In other aspects, the nucleic acid encoding any of the IDI polypeptides described herein can be integrated into the chromosome of the host cell. In some aspects, the IDI nucleic acid is operably linked to a constitutive promoter. In other aspects, the IDI nucleic acid is operably linked to an inducible promoter.

Exemplary DXP Pathway Polypeptides and Nucleic Acids

Various DXP pathway polypeptides can be used to increase the flow of carbon through the DXP pathway, leading to greater isoprene production. Exemplary DXP pathway polypeptides include polypeptides, fragments of polypeptides, peptides, and fusions polypeptides that have at least one activity of a DXP pathway polypeptide. In one aspect, DXS polypeptide can be used to increase the flow of carbon through the DXP pathway, leading to greater isoprene production. Standard methods known to one of skill in the art and as taught the references cited herein can be used to determine whether a polypeptide has DXS polypeptide activity by measuring the ability of the polypeptide to convert pyruvate and D-glyceraldehyde-3-phosphate into 1-deoxy-D-xylulose-5-phosphate in vitro, in a cell extract, or in vivo. Exemplary DXS nucleic acids include nucleic acids that encode a polypeptide, fragment of a polypeptide, peptide, or fusion polypeptide that has at least one activity of a DXS polypeptide. Exemplary DXS polypeptides and nucleic acids include naturally-occurring polypeptides and nucleic acids from any of the source organisms described herein as well as mutant polypeptides and nucleic acids derived from any of the source organisms described herein. Exemplary DXS polypeptides and nucleic acids and methods of measuring DXS activity are described in more detail in International Patent Application Publication Nos. WO 2009/076676, U.S. patent application Ser. No. 12/335,071 (US Publ. No. 2009/0203102), WO 2010/003007, US Publ. No. 2010/0048964, WO 2009/132220, and US Publ. No. 2010/0003716.

Exemplary DXP pathways polypeptides that can be used include, but are not limited to any of the following polypeptides: DXS polypeptides, DXR polypeptides, MCT polypeptides, CMK polypeptides, MCS polypeptides, HDS polypeptides, HDR polypeptides, and polypeptides (e.g., fusion polypeptides) having an activity of one, two, or more of the DXP pathway polypeptides. In particular, DXP pathway polypeptides include polypeptides, fragments of polypeptides, peptides, and fusions polypeptides that have at least one activity of a DXP pathway polypeptide. Exemplary DXP pathway nucleic acids include nucleic acids that encode a polypeptide, fragment of a polypeptide, peptide, or fusion polypeptide that has at least one activity of a DXP pathway polypeptide. Exemplary DXP pathway polypeptides and nucleic acids include naturally-occurring polypeptides and nucleic acids from any of the source organisms described herein as well as mutant polypeptides and nucleic acids derived from any of the source organisms described herein. Exemplary DXP pathway polypeptides and nucleic acids and methods of measuring DXP pathway polypeptide activity are described in more detail in International Patent Application Publication No.: WO 2010/148150.

In particular, DXS polypeptides convert pyruvate and D-glyceraldehyde 3-phosphate into 1-deoxy-d-xylulose 5-phosphate (DXP). Standard methods can be used to determine whether a polypeptide has DXS polypeptide activity by measuring the ability of the polypeptide to convert pyruvate and D-glyceraldehyde 3-phosphate in vitro, in a cell extract, or in vivo.

DXR polypeptides convert 1-deoxy-d-xylulose 5-phosphate (DXP) into 2-C-methyl-D-erythritol 4-phosphate (MEP). Standard methods can be used to determine whether a polypeptide has DXR polypeptides activity by measuring the ability of the polypeptide to convert DXP in vitro, in a cell extract, or in vivo.

MCT polypeptides convert 2-C-methyl-D-erythritol 4-phosphate (MEP) into 4-(cytidine 5′-diphospho)-2-methyl-D-erythritol (CDP-ME). Standard methods can be used to determine whether a polypeptide has MCT polypeptides activity by measuring the ability of the polypeptide to convert MEP in vitro, in a cell extract, or in vivo.

CMK polypeptides convert 4-(cytidine 5′-diphospho)-2-C-methyl-D-erythritol (CDP-ME) into 2-phospho-4-(cytidine 5′-diphospho)-2-C-methyl-D-erythritol (CDP-MEP). Standard methods can be used to determine whether a polypeptide has CMK polypeptides activity by measuring the ability of the polypeptide to convert CDP-ME in vitro, in a cell extract, or in vivo.

MCS polypeptides convert 2-phospho-4-(cytidine 5′-diphospho)-2-C-methyl-D-erythritol (CDP-MEP) into 2-C-methyl-D-erythritol 2,4-cyclodiphosphate (ME-CPP or cMEPP). Standard methods can be used to determine whether a polypeptide has MCS polypeptides activity by measuring the ability of the polypeptide to convert CDP-MEP in vitro, in a cell extract, or in vivo.

HDS polypeptides convert 2-C-methyl-D-erythritol 2,4-cyclodiphosphate into (E)-4-hydroxy-3-methylbut-2-en-1-yl diphosphate (HMBPP or HDMAPP). Standard methods can be used to determine whether a polypeptide has HDS polypeptides activity by measuring the ability of the polypeptide to convert ME-CPP in vitro, in a cell extract, or in vivo.

HDR polypeptides convert (E)-4-hydroxy-3-methylbut-2-en-1-yl diphosphate into isopentenyl diphosphate (IPP) and dimethylallyl diphosphate (DMAPP). Standard methods can be used to determine whether a polypeptide has HDR polypeptides activity by measuring the ability of the polypeptide to convert HMBPP in vitro, in a cell extract, or in vivo.

In some embodiments, the DXP pathway polypeptide is an endogenous polypeptide. In some embodiments, the cells comprise one or more additional copies of an endogenous nucleic acid encoding a DXP pathway polypeptide. In other embodiments, the DXP pathway polypeptide is a heterologous polypeptide. In some embodiments, the cells comprise more than one copy of a heterologous nucleic acid encoding a DXP pathway polypeptide. In any of the embodiments herein, the nucleic acid is operably linked to a promoter (e.g., inducible or constitutive promoter).

Source Organisms

Isoprene synthase, IDI, MVA pathway nucleic acids (and their encoded polypeptides) and/or DXP pathway nucleic acids (and their encoded polypeptides) can be obtained from any organism that naturally contains isoprene synthase and/or MVA pathway nucleic acids and/or DXP pathway nucleic acids. As noted above, isoprene is formed naturally by a variety of organisms, such as bacteria, yeast, plants, and animals. Some organisms contain the MVA pathway for producing isoprene. Isoprene synthase nucleic acids can be obtained, e.g., from any organism that contains an isoprene synthase. IDI nucleic acids can be obtained, e.g., from any organisms that contains an IDI. MVA pathway nucleic acids can be obtained, e.g., from any organism that contains the MVA pathway. DXP pathway nucleic acids can be obtained, e.g., from any organism that contains the DXP pathway.

Exemplary sources for isoprene synthases, MVA pathway polypeptides, and/or DXP pathway polypeptides and other polypeptides (including nucleic acids encoding any of the polypeptides described herein) which can be used are also described in International Patent Application Publication No. WO2009/076676; and U.S. Patent Application Publication Nos. 2010/0048964, 2010/0086978, 2010/0167370, 2010/0113846, 2010/0184178, 2010/0167371, 2010/0196977, 2011/0014672, and 2011/0046422.

### Host Cell Mutations to Improve Acetate Utilization

There are two alternative pathways for acetate utilization in Escherichia coli (Gimenez et al, 2003, J Bacteriol. 185: 6448-6455). One of these pathways is mediated by acetyl coenzyme A (acetyl-CoA) synthetase (EC 6.2.1.1), which catalyzes acetyl-CoA formation through an enzyme-bound acetyladenylate intermediate in an irreversible reaction. This enzyme is encoded by the gene acs. In certain aspects, the acs gene activity can be increased by standard molecular biology techniques (e.g., via the use of a strong or constitutive promoter) to improve acetate utilization in the host cells used in the methods described herein. In certain embodiments, the amount of acs gene activity is increased such that it can be at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The other pathway, mediated by the enzymes acetate kinase and phosphotransacetylase, proceeds through a high-energy acetyl phosphate intermediate in two reversible reactions. These two enzymes are encoded by the ackA and pta genes. Phosphotransacetylase (pta) (Shimizu et al., 1969. Biochim. Biophys. Acta 191: 550-558) catalyzes the reversible conversion between acetyl-CoA and acetylphosphate (acetyl-P), while acetate kinase (ackA) (Kakuda, H. et al., 1994. J. Biochem. 11:916-922) uses acetyl-P to form acetate. These genes can be transcribed as an operon in E. coli. In certain aspects, the activity ackA and/or pta can be increased by standard molecular biology techniques (e.g., via the use of a strong or constitutive promoter) to improve acetate utilization in the host cells used in the methods described herein. In certain embodiments, the amount of ackA and/or pta gene activity is increased such that it can be at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The aceBAK operon encodes for the enzymes of the glyoxylate bypass that are required during growth on acetate since it bypasses the two CO2-evolving steps of the Krebs cycle (Lorca et al., 2007, J. Biological Chemistry 282:16476-16491). The expression of these enzymes is typically induced during growth on minimal medium supplemented with acetate or fatty acids as well as in rich medium as a result of the acetate accumulation during exponential phase. In certain aspects, the activity aceBAK operon can be increased by standard molecular biology techniques (e.g., via the use of a strong or constitutive promoter) to improve acetate utilization in the host cells used in the methods described herein. In certain embodiments, the amount of aceBAK operon activity is increased such that it can be at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The pts operon of Escherichia coli is composed of the ptsH, ptsI and crr genes coding for three proteins central to the phosphoenolpyruvate dependent phosphotransferase system (PTS), the HPr, enzyme I and EIIIGlc proteins. These three genes are organized in a complex operon in which the major part of expression of the distal gene, crr, is initiated from a promoter region within ptsI. Transcription from this promoter region is under the positive control of catabolite activator protein (CAP)-cyclic AMP (cAMP) and is enhanced during growth in the presence of glucose (a PTS substrate). In certain embodiments described herein, the down regulation (e.g. attenuation) of the pts operon can enhance acetate utilization by the host cells. The down regulation of PTS operon activity can be any amount of reduction of specific activity or total activity as compared to when no manipulation has been effectuated. In some instances, the decrease of activity of the complex is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

### Additional Host Cell Mutations

The invention also contemplates using additional host cell mutations that increase carbon flux through the MVA pathway. By increasing the carbon flow, more isoprene can be produced. The recombinant cells as described herein can also be engineered for increased carbon flux towards mevalonate production wherein the activity of one or more enzymes from the group consisting of: (a) citrate synthase, (b) phosphotransacetylase; (c) acetate kinase; (d) lactate dehydrogenase; (e) NADP-dependent malic enzyme, and; (f) pyruvate dehydrogenase is modulated.

Citrate Synthase Pathway

Citrate synthase catalyzes the condensation of oxaloacetate and acetyl-CoA to form citrate, a metabolite of the Tricarboxylic acid (TCA) cycle (Ner, S. et al. 1983. Biochemistry 22: 5243-5249; Bhayana, V. and Duckworth, H. 1984. Biochemistry 23: 2900-2905). In E. coli, this enzyme, encoded by gltA, behaves like a trimer of dimeric subunits. The hexameric form allows the enzyme to be allosterically regulated by NADH. This enzyme has been widely studied (Wiegand, G., and Remington, S. 1986. Annual Rev. Biophysics Biophys. Chem., 15: 97-117; Duckworth et al. 1987. Biochem Soc Symp. 54:83-92; Stockell, D. et al., 2003. J. Biol. Chem. 278: 35435-43; Maurus, R. et al. 2003. Biochemistry. 42:5555-5565). To avoid allosteric inhibition by NADH, replacement by or supplementation with the Bacillus subtilis NADH-insensitive citrate synthase has been considered (Underwood et al., 2002. Appl. Environ. Microbiol. 68:1071-1081; Sanchez et al., 2005, Met. Eng. 7:229-239).

The reaction catalyzed by citrate synthase directly competes with the thiolase catalyzing the first step of the mevalonate pathway, as they both have acetyl-CoA as a substrate (Hedl et al., 2002, J. Bact. 184:2116-2122). Therefore, one of skill in the art can modulate citrate synthase expression (e.g., decrease enzyme activity) to allow more carbon to flux into the mevalonate pathway, thereby increasing the eventual production of mevalonate or isoprene. The decrease of citrate synthase activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%. In some aspects, the activity of citrate synthase is modulated by decreasing the activity of an endogenous citrate synthase gene. This can be accomplished by chromosomal replacement of an endogenous citrate synthase gene with a transgene encoding an NADH-insensitive citrate synthase or by using a transgene encoding an NADH-insensitive citrate synthase that is derived from Bacillus subtilis. The activity of citrate synthase can also be modulated (e.g., decreased) by replacing the endogenous citrate synthase gene promoter with a synthetic constitutively low expressing promoter. The decrease of the activity of citrate synthase can result in more carbon flux into the mevalonate dependent biosynthetic pathway in comparison to microorganisms that do not have decreased expression of citrate synthase.

Pathways Involving Phosphotransacetylase and/or Acetate Kinase

Phosphotransacetylase (pta) (Shimizu et al., 1969. Biochim. Biophys. Acta 191: 550-558) catalyzes the reversible conversion between acetyl-CoA and acetylphosphate (acetyl-P), while acetate kinase (ackA) (Kakuda, H. et al., 1994. J. Biochem. 11:916-922) uses acetyl-P to form acetate. These genes can be transcribed as an operon in E. coli. Together, they catalyze the dissimilation of acetate with the release of ATP. Thus, one of skill in the art can increase the amount of available acetyl Co-A by attenuating the activity of phosphotransacetylase gene (e.g., the endogenous phosphotransacetylase gene) and/or an acetate kinase gene (e.g., the endogenous acetate kinase gene). One way of achieving attenuation is by deleting phosphotransacetylase (pta) and/or acetate kinase (ackA). This can be accomplished, for example, by replacing one or both genes with a chloramphenicol cassette followed by looping out of the cassette. Acetate is produced by E. coli for a variety of reasons (Wolfe, A. 2005. Microb. Mol. Biol. Rev. 69:12-50). Without being bound by theory, since ackA-pta use acetyl-CoA, deleting those genes might allow carbon not to be diverted into acetate and to increase the yield of mevalonate or isoprene.

In some aspects, the recombinant microorganism produces decreased amounts of acetate in comparison to microorganisms that do not have attenuated endogenous phosphotransacetylase gene and/or endogenous acetate kinase gene expression. Decrease in the amount of acetate produced can be measured by routine assays known to one of skill in the art. The amount of acetate reduction is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of phosphotransacetylase (pta) and/or acetate kinase (ackA) can also be decreased by other molecular manipulation of the enzymes. The decrease of enzyme activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

In some cases, attenuating the activity of the endogenous phosphotransacetylase gene and/or the endogenous acetate kinase gene results in more carbon flux into the mevalonate dependent biosynthetic pathway in comparison to microorganisms that do not have attenuated endogenous phosphotransacetylase gene and/or endogenous acetate kinase gene expression.

Pathways Involving Lactate Dehydrogenase

In E. coli, D-Lactate is produced from pyruvate through the enzyme lactate dehydrogenase (ldhA) (Bunch, P. et al. 1997. Microbiol. 143:187-195). Production of lactate is accompanied by oxidation of NADH, hence lactate is produced when oxygen is limited and cannot accommodate all the reducing equivalents. Thus, production of lactate could be a source of carbon consumption. As such, to improve carbon flow through to mevalonate production and isoprene production, one of skill in the art can modulate the activity of lactate dehydrogenase, such as by decreasing the activity of the enzyme.

Accordingly, in one aspect, the activity of lactate dehydrogenase can be modulated by attenuating the activity of an endogenous lactate dehydrogenase gene. Such attenuation can be achieved by deletion of the endogenous lactate dehydrogenase gene. Other ways of attenuating the activity of lactate dehydrogenase gene known to one of skill in the art may also be used. By manipulating the pathway that involves lactate dehydrogenase, the recombinant microorganism produces decreased amounts of lactate in comparison to microorganisms that do not have attenuated endogenous lactate dehydrogenase gene expression. Decrease in the amount of lactate produced can be measured by routine assays known to one of skill in the art. The amount of lactate reduction is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared to when no molecular manipulations are done.

The activity of lactate dehydrogenase can also be decreased by other molecular manipulations of the enzyme. The decrease of enzyme activity can be any amount of reduction of specific activity or total activity as compared to when no manipulation has been effectuated. In some instances, the decrease of enzyme activity is decreased by at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

Accordingly, in some cases, attenuation of the activity of the endogenous lactate dehydrogenase gene results in more carbon flux into the mevalonate dependent biosynthetic pathway in comparison to microorganisms that do not have attenuated endogenous lactate dehydrogenase gene expression.

Pathways Involving Malic Enzyme

Malic enzyme (in E. coli sfcA and maeB) is an anaplerotic enzyme that catalyzes the conversion of malate into pyruvate (using NAD+ or NADP+) by the equation below:

(S)-malate+NAD(P)⁺ [symbol] pyruvate+CO₂+NAD(P)H

Thus, the two substrates of this enzyme are (S)-malate and NAD(P)⁺, whereas its 3 products are pyruvate, CO₂, and NADPH.

Expression of the NADP-dependent malic enzyme (maeB) (Iwikura, M. et al. 1979. J. Biochem. 85: 1355-1365) can help increase mevalonate and isoprene yield by 1) bringing carbon from the TCA cycle back to pyruvate, direct precursor of acetyl-CoA, itself direct precursor of the mevalonate pathway and 2) producing extra NADPH which could be used in the HMG-CoA reductase reaction (Oh, M K et al. (2002) J. Biol. Chem. 277: 13175-13183; Bologna, F. et al. (2007) J. Bact. 189:5937-5946).

As such, more starting substrate (pyruvate or acetyl-CoA) for the downstream production of mevalonate and isoprene can be achieved by modulating, such as increasing, the activity and/or expression of malic enzyme. The NADP-dependent malic enzyme gene can be an endogenous gene. One non-limiting way to accomplish this is by replacing the endogenous NADP-dependent malic enzyme gene promoter with a synthetic constitutively expressing promoter. Another non-limiting way to increase enzyme activity is by using one or more heterologous nucleic acids encoding an NADP-dependent malic enzyme polypeptide. One of skill in the art can monitor the expression of maeB RNA during fermentation or culturing using readily available molecular biology techniques.

Accordingly, in some embodiments, the recombinant microorganism produces increased amounts of pyruvate in comparison to microorganisms that do not have increased expression of an NADP-dependent malic enzyme gene. In some aspects, increasing the activity of an NADP-dependent malic enzyme gene results in more carbon flux into the mevalonate dependent biosynthetic pathway in comparison to microorganisms that do not have increased NADP-dependent malic enzyme gene expression.

Increase in the amount of pyruvate produced can be measured by routine assays known to one of skill in the art. The amount of pyruvate increase can be at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99% as compared when no molecular manipulations are done.

The activity of malic enzyme can also be increased by other molecular manipulations of the enzyme. The increase of enzyme activity can be any amount of increase of specific activity or total activity as compared to when no manipulation has been effectuated. In some instances, the increase of enzyme activity is at least about 1%, 2%, 3%, 4%, 5%, 6%, 7%, 8%, 9%, 10%, 15%, 20%, 25%, 30%, 35%, 35%, 40%, 45%, 50%, 55%, 60%, 65%, 70%, 75%, 80%, 85%, 90%, 95%, 96%, 97%, 98%, or 99%.

Pathways Involving Pyruvate Dehydrogenase Complex

The pyruvate dehydrogenase complex, which catalyzes the decarboxylation of pyruvate into acetyl-CoA, is composed of the proteins encoded by the genes aceE, aceF and lpdA. Transcription of those genes is regulated by several regulators. Thus, one of skill in the art can increase acetyl-CoA by modulating the activity of the pyruvate dehydrogenase complex. Modulation can be to increase the activity and/or expression (e.g., constant expression) of the pyruvate dehydrogenase complex. This can be accomplished by different ways, for example, by placing a strong constitutive promoter, like PL.6 (aattcatataaaaaacatacagataaccatctgcggtgataaattatctctggcggtgttgacataaataccactggcggtgatactgagcac atcagcaggacgcactgaccaccatgaaggtg-lambda promoter, GenBank NC_{—}001416), in front of the operon or using one or more synthetic constitutively expressing promoters.

Accordingly, in one aspect, the activity of pyruvate dehydrogenase is modulated by increasing the activity of one or more genes of the pyruvate dehydrogenase complex consisting of (a) pyruvate dehydrogenase (E1), (b) dihydrolipoyl transacetylase, and (c) dihydrolipoyl dehydrogenase. It is understood that any one, two or three of these genes can be manipulated for increasing activity of pyruvate dehydrogenase. In another aspect, the activity of the pyruvate dehydrogenase complex can be modulated by attenuating the activity of an endogenous pyruvate dehydrogenase complex repressor gene, further detailed below. The activity of an endogenous pyruvate dehydrogenase complex repressor can be attenuated by deletion of the endogenous pyruvate dehydrogenase complex repressor gene.

In some cases, one or more genes of the pyruvate dehydrogenase complex are endogenous genes. Another way to increase the activity of the pyruvate dehydrogenase complex is by introducing into the microorganism one or more heterologous nucleic acids encoding one or more polypeptides from the group consisting of (a) pyruvate dehydrogenase (E1), (b) dihydrolipoyl transacetylase, and (c) dihydrolipoyl dehydrogenase.

By using any of these methods, the recombinant microorganism can produce increased amounts of acetyl Co-A in comparison to microorganisms wherein the activity of pyruvate dehydrogenase is not modulated. Modulating the activity of pyruvate dehydrogenase can result in more carbon flux into the mevalonate dependent biosynthetic pathway in comparison to microorganisms that do not have modulated pyruvate dehydrogenase expression.

Combinations of Mutations

It is understood that for any of the enzymes and/or enzyme pathways described herein, molecular manipulations that modulate any combination (such as two, three, four, five or six) of the enzymes and/or enzyme pathways described herein is expressly contemplated. For ease of the recitation of the combinations, citrate synthase (gltA) is designated as A, phosphotransacetylase (ptaB) is designated as B, acetate kinase (ackA) is designated as C, lactate dehydrogenase (ldhA) is designated as D, malic enzyme (sfcA or maeB) is designated as E, and pyruvate decarboxylase (aceE, aceF, and/or lpdA) is designated as F. As discussed above, aceE, aceF, and/or lpdA enzymes of the pyruvate decarboxylase complex can be used singly, or two of three enzymes, or three of three enzymes for increasing pyruvate decarboxylase activity.

Accordingly, for combinations of any two of the enzymes A-F, non-limiting combinations that can be used are: AB, AC, AD, AE, AF, BC, BD, BE, BF, CD, CE, CF, DE, DF and EF. For combinations of any three of the enzymes A-F, non-limiting combinations that can be used are: ABC, ABD, ABE, ABF, BCD, BCE, BCF, CDE, CDF, DEF, ACD, ACE, ACF, ADE, ADF, AEF, BDE, BDF, BEF, and CEF. For combinations of any four of the enzymes A-F, non-limiting combinations that can be used are: ABCD, ABCE, ABCF, ABDE, ABDF, ABEF, BCDE, BCDF, CDEF, ACDE, ACDF, ACEF, BCEF, BDEF, and ADEF. For combinations of any five of the enzymes A-F, non-limiting combinations that can be used are: ABCDE, ABCDF, ABDEF, BCDEF, ACDEF, and ABCEF. In another aspect, all six enzyme combinations are used: ABCDEF.

Accordingly, the recombinant microorganism as described herein can achieve increased mevalonate production that is increased compared to microorganisms that are not grown under conditions of tri-carboxylic acid (TCA) cycle activity, wherein metabolic carbon flux in the recombinant microorganism is directed towards mevalonate production by modulating the activity of one or more enzymes from the group consisting of (a) citrate synthase, (b) phosphotransacetylase and/or acetate kinase, (c) lactate dehydrogenase, (d) malic enzyme, and (e) pyruvate decarboxylase complex.

Other Regulators and Factors for Increased Production

Other molecular manipulations can be used to increase the flow of carbon towards mevalonate and/or isoprene production. One method is to reduce, decrease or eliminate the effects of negative regulators for pathways that feed into the mevalonate pathway. For example, in some cases, the genes aceEF-lpdA are in an operon, with a fourth gene upstream pdhR. pdhR is a negative regulator of the transcription of its operon. In the absence of pyruvate, it binds its target promoter and represses transcription. It also regulates ndh and cyoABCD in the same way (Ogasawara, H. et al. 2007. J. Bact. 189:5534-5541). In one aspect, deletion of pdhR regulator can improve the supply of pyruvate, and hence the production of mevalonate and isoprene.

In other aspects, the introduction of 6-phosphogluconolactonase (PGL) into microorganisms (such as various E. coli strains) which lack PGL can be used to improve production of mevalonate and isoprene. PGL may be introduced using chromosomal integration or extra-chromosomal vehicles, such as plasmids.

### Vectors

One of skill in the art will recognize that expression vectors are designed to contain certain components which optimize gene expression for certain host strains. Such optimization components include, but are not limited to origin of replication, promoters, and enhancers. The vectors and components referenced herein are described for exemplary purposes and are not meant to narrow the scope of the invention.

Suitable vectors can be used to express any of the above polypeptides in isoprene, isoprenoid, and isoprenoid precursor-producing cells in any of the methods described herein. For example, suitable vectors can be used to optimize the expression of one or more copies of a gene encoding an isoprene synthase, IDI, polyprenyl pyrophosphate synthase, DXP pathway polypeptides, and/or MVA pathway nucleic acid(s) and/or DXP pathway nucleic acid(s) in anaerobes. In some aspects, the vector contains a selective marker. Examples of selectable markers include, but are not limited to, antibiotic resistance nucleic acids (e.g., kanamycin, ampicillin, carbenicillin, gentamicin, hygromycin, phleomycin, bleomycin, neomycin, or chloramphenicol) and/or nucleic acids that confer a metabolic advantage, such as a nutritional advantage on the host cell. In some aspects, one or more copies of an isoprene synthase, IDI, polyprenyl pyrophosphate synthase, and/or MVA pathway nucleic acid(s) and/or DXP pathway nucleic acid(s) integrate into the genome of host cells without a selective marker.

In some aspects, the vector is a shuttle vector, which is capable of propagating in two or more different host species. Exemplary shuttle vectors are able to replicate in E. coli and/or Bacillus subtilis and in an obligate anaerobe, such as Clostridium. Upon insertion of an isoprene synthase or MVA pathway nucleic acid into the shuttle vector using techniques well known in the art, the shuttle vector can be introduced into an E. coli host cell for amplification and selection of the vector. The vector can then be isolated and introduced into an obligate anaerobic cell for expression of the isoprene synthase or MVA pathway polypeptide.

Any one of the vectors characterized or used in the Examples of the present disclosure can also be used.

### Host Cells

Various types of host cells can be used to produce mevalonate, isoprenoid precursor molecules, isoprene, and/or isoprenoids in any of the methods described herein.

In some aspects, the host cell is a yeast, such as Saccharomyces sp., Schizosaccharomyces sp., Pichia sp., Candida sp. or Yarrowia (such as, Y. lipolytica). In some aspects, the Saccharomyces sp. is Saccharomyces cerevisiae (See, e.g., Romanos et al., Yeast, (1992), 8(6):423-488). In some aspects, the yeast cells are Yarrowia lipolytica cells. In certain embodiments, plasmids or plasmid components for use herein include those described in U.S. Pat. No. 7,659,097 and U.S. Patent Pub. No. US 2011/0045563.

In some aspects, the host cell is a bacterium, such as strains of Bacillus such as B. lichenformis or B. subtilis, strains of Pantoea such as P. citrea, strains of Pseudomonas such as P. alcaligenes, strains of Streptomyces such as S. lividans or S. rubiginosus, strains of Escherichia such as E. coli, strains of Enterobacter, strains of Streptococcus, strains of Corynebacterium such as C. glutamicum, or strains of Archaea such as Methanosarcina mazei.

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. Co-Agulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In some aspects, the host cell is a gram-positive bacterium. Non-limiting examples include strains of Streptomyces (e.g., S. lividans, S. coelicolor, or S. griseus) and Bacillus (such as, but not limited to, B. subtilis) Listeria (e.g., L. monocytogenes) or Lactobacillus (e.g., L. spp). In some aspects, the source organism is a gram-negative bacterium, such as a member of Escherichia sp. (e.g., E. coli), Pantoea sp. (e.g., P. citrea), or Pseudomonas sp.

In some aspects, the host cell is a plant, such as a plant from the family Fabaceae, such as the Faboideae subfamily. In some aspects, the host cell is kudzu, poplar (such as Populus alba×tremula CAC35696), aspen (such as Populus tremuloides), or Quercus robur.

In some aspects, the host cell is a fungus. In certain aspects, the host cell can be a filamentous fungal cell and progeny thereof. (See, e.g., Berka & Barnett, Biotechnology Advances, (1989), 7(2):127-154). In some aspects, the filamentous fungal cell can be any of Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp., such as A. oryzae, A. niger, A sojae, A. japonicus, A. nidulans, or A. awamori, Fusarium sp., such as F. roseum, F. graminum F. cerealis, F. oxysporuim, or F. venenatum, Neurospora sp., such as N. crassa, Hypocrea sp., Mucor sp., such as M. miehei, Rhizopus sp. or Emericella sp. In some aspects, the fungus is A. nidulans, A. awamori, A. oryzae, A. aculeatus, A. niger, A. japonicus, T. reesei, T. viride, F. oxysporum, or F. solani. In certain embodiments, plasmids or plasmid components for use herein include those described in U.S. Patent Pub. No. US 2011/0045563. In some aspects the host cell is a member of the Trichoderma sp. In other aspects, the host cell is T. reesei.

The host cell can additionally be a species of algae, such as a green algae, red algae, glaucophytes, chlorarachniophytes, euglenids, chromista, or dinoflagellates. (See, e.g., Saunders & Warmbrodt, “Gene Expression in Algae and Fungi, Including Yeast,” (1993), National Agricultural Library, Beltsville, Md.). In certain embodiments, plasmids or plasmid components for use herein include those described in U.S. Patent Pub. No. US 2011/0045563.

In some aspects, the host cell is a cyanobacterium, such as cyanobacterium classified into any of the following groups based on morphology: Chlorococcales, Pleurocapsales, Oscillatoriales, Nostocales, or Stigonematales (See, e.g., Lindberg et al., Metab. Eng., (2010) 12(1):70-79). In certain embodiments, plasmids or plasmid components for use herein include those described in U.S. Patent Pub. No. US 2010/0297749 and US 2009/0282545 and Intl. Pat. Appl. No. WO 2011/034863.

In some aspects, the host cell is an anaerobic organism. An “anaerobe” is an organism that does not require oxygen for growth. An anaerobe can be an obligate anaerobe, a facultative anaerobe, or an aerotolerant organism. Such organisms can be any of the organisms listed above, bacteria, yeast, etc. An “obligate anaerobe” is an anaerobe for which atmospheric levels of oxygen can be lethal. Examples of obligate anaerobes include, but are not limited to, Clostridium, Eurobacterium, Bacteroides, Peptostreptococcus, Butyribacterium, Veillonella, and Actinomyces. In one aspect, the obligate anaerobes can be any one or combination selected from the group consisting of Clostridium ljungdahlii, Clostridium autoethanogenum, Eurobacterium limosum, Clostridium carboxydivorans, Peptostreptococcus productus, and Butyribacterium methylotrophicum. A “facultative anaerobe” is an anaerobe that is capable of performing aerobic respiration in the presence of oxygen and is capable of performing anaerobic fermentation under oxygen-limited or oxygen-free conditions. Examples of facultative anaerobes include, but are not limited to, Escherichia, Pantoea, yeast, and Yarrowia.

In some aspects, the host cell is a photosynthetic cell. In other aspects, the host cell is a non-photosynthetic cell.

### Transformation Methods

Nucleic acids encoding an isoprene synthase, IDI, polyprenyl pyrophosphate synthase, and/or MVA pathway nucleic acid(s) and/or DXP pathway nucleic acid(s) can be inserted into any host cell using standard techniques. General transformation techniques are known in the art (see, e.g., Current Protocols in Molecular Biology (F. M. Ausubel et al. (eds) Chapter 9, 1987; Sambrook et al., Molecular Cloning: A Laboratory Manual, 2^{nd} ed., Cold Spring Harbor, 1989; and Campbell et al., Curr. Genet. 16:53-56, 1989 or “Handbook on Clostridia” (P. Dune, ed., 2004). For obligate anaerobic host cells, such as Clostridium, electroporation, as described by Davis et al., 2005 and in Examples III and IV, can be used as an effective technique. The introduced nucleic acids may be integrated into chromosomal DNA or maintained as extrachromosomal replicating sequences.

### Cell Culture Media

As used herein, the terms “minimal medium” or “minimal media” refer to growth medium containing the minimum nutrients possible for cell growth, generally, but not always, without the presence of one or more amino acids (e.g., 1, 2, 3, 4, 5, 6, 7, 8, 9, 10, or more amino acids). Minimal medium typically contains: (1) a carbon source for microbial (e.g., bacterial) growth; (2) various salts, which may vary among microbial (e.g., bacterial) species and growing conditions; and (3) water. The carbon source can vary significantly, from simple sugars like glucose to more complex hydrolysates of other biomass, such as yeast extract, as discussed in more detail below. The salts generally provide essential elements such as magnesium, nitrogen, phosphorus, and sulfur to allow the cells to synthesize proteins and nucleic acids. Minimal medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

Any minimal medium formulation can be used to cultivate the host cells. Exemplary minimal medium formulations include, for example, M9 minimal medium and TM3 minimal medium. Each liter of M9 minimal medium contains (1) 200 ml sterile M9 salts (64 g Na₂HPO₄-7H₂O, 15 g KH₂PO₄, 2.5 g NaCl, and 5.0 g NH₄Cl per liter); (2) 2 ml of 1 M MgSO₄ (sterile); (3) 20 ml of 20% (w/v) glucose (or other carbon source); and (4) 100 μl of 1 M CaCl₂ (sterile). Each liter of TM3 minimal medium contains (1) 13.6 g K₂HPO₄; (2) 13.6 g KH₂PO₄; (3) 2 g MgSO₄*7H₂O; (4) 2 g Citric Acid Monohydrate; (5) 0.3 g Ferric Ammonium Citrate; (6) 3.2 g (NH₄)₂SO₄; (7) 0.2 g yeast extract; and (8) 1 ml of 1000× Trace Elements solution; pH is adjusted to ˜6.8 and the solution is filter sterilized. Each liter of 1000× Trace Elements contains: (1) 40 g Citric Acid Monohydrate; (2) 30 g MnSO₄*H₂O; (3) 10 g NaCl; (4) 1 g FeSO₄*7H₂O; (4)1 g CoCl₂*6H₂O; (5) 1 g ZnSO₄*7H₂O; (6) 100 mg CuSO₄*5H₂O; (7) 100 mg H₃BO₃; and (8) 100 mg NaMoO₄*2H₂O; pH is adjusted to ˜3.0.

Any carbon source can be used to cultivate the host cells. The term “carbon source” refers to one or more carbon-containing compounds capable of being metabolized by a host cell or organism. For example, the cell medium used to cultivate the host cells may include any carbon source suitable for maintaining the viability or growing the host cells. In some aspects, the carbon source is a carbohydrate (such as monosaccharide, disaccharide, oligosaccharide, or polysaccharides), or invert sugar (e.g., enzymatically treated sucrose syrup).

Exemplary monosaccharides include glucose and fructose; exemplary oligosaccharides include lactose and sucrose, and exemplary polysaccharides include starch and cellulose. Exemplary carbohydrates include C6 sugars (e.g., fructose, mannose, galactose, or glucose) and C5 sugars (e.g., xylose or arabinose).

In some aspects, the media used to cultivate any of the engineered cells in any of the methods disclosed herein contains a carbon source. In some aspects, the culture media comprises both a carbon source (such as glucose) and acetate. Any media (including, for example, M9 minimal medium and/or TM3 minimal media), can be supplemented with glucose and acetate. In some aspects, the media contains any of about 0.1%, 0.2%, 0.3%, 0.4%, 0.5%, 0.6%, 0.7%, 0.8%, 0.9%, 1.0%, 1.1%, 1.2%, 1.3%, 1.4%, or 1.5% glucose, inclusive, including any numbers between these percentages. In other aspects, the media contains any of about 0.05%, 0.1%, 0.2%, 0.25%, 0.3%, 0.4%, 0.5%, 0.6%, 0.7%, 0.8%, 0.9%, 1.0%, 1.1%, 1.2%, 1.3%, 1.4%, or 1.5% acetate, inclusive, including any numbers between these percentages. In some aspects, the cells can be cultured in media having a 1% glucose concentration and a concentration of acetate of at least about 0.01% to about 1.5%. In other aspects, the concentration of acetate is at least about 0.01% to about 0.75%, at least about 0.01% to about 0.5%, at least about 0.01% to about 0.4%, at least about 0.01% to about 0.3%, at least about 0.01% to about 0.25%, or at least about 0.01% to about 0.2%. In some aspects, the acetate concentration of the media is 0.5%.

In certain embodiments, other alternate more oxidized substrates can be substituted in place of acetate in the methods described herein to obtain increased production of intracellular acetyl-CoA concentrations, mevalonate, isoprenoid precursors, isoprene and/or isoprenoids. These alternate more oxidized substrates include, but are not limited to, citrate, butyrate, propionate, and TCA-intermediates (e.g., α-ketoglutarate and gluconate).

Co-Culturing Recombinant Cells in Parallel with Homoacetogenic Microorganisms

Homoacetogens are a versatile family of mostly anaerobic bacteria that are able to convert a variety of different substrates to acetate as a major end product. Most homoacetogens grow using hydrogen plus CO₂ as their sole energy source. Hydrogen provides electrons for the reduction of CO₂ to acetate. The methyl group of acetate is generated from CO₂ via formate and reduced C1 intermediates bound to tetrahydrofolate. The carboxyl group is derived from carbon monoxide by the enzyme carbon monoxide dehydrogenase. This enzyme additionally catalyzes the formation of acetyl-CoA from methyl groups plus carbon monoxide. Acetyl-CoA is then converted either to acetate during catabolism or to carbon during anabolism.

The acetate used in any of the media described above can come from any source. In one aspect, acetate can be obtained from homoacetogenic microorganisms such as, but not limited to, Clostridia. The biological conversion of syngas to acetate by homoacetogenic microorganisms has been demonstrated with a near 100% yield (See, e.g., Morinaga and Kawada, Journal of Biotechnology, 14: 187-194 (1990). Consequently, in some aspects, acetate used for the culturing of microorganisms via the co-metabolism of glucose and acetate in any of the methods described herein can be obtained from the fermentation of syngas using homoacetogenic microorganisms.

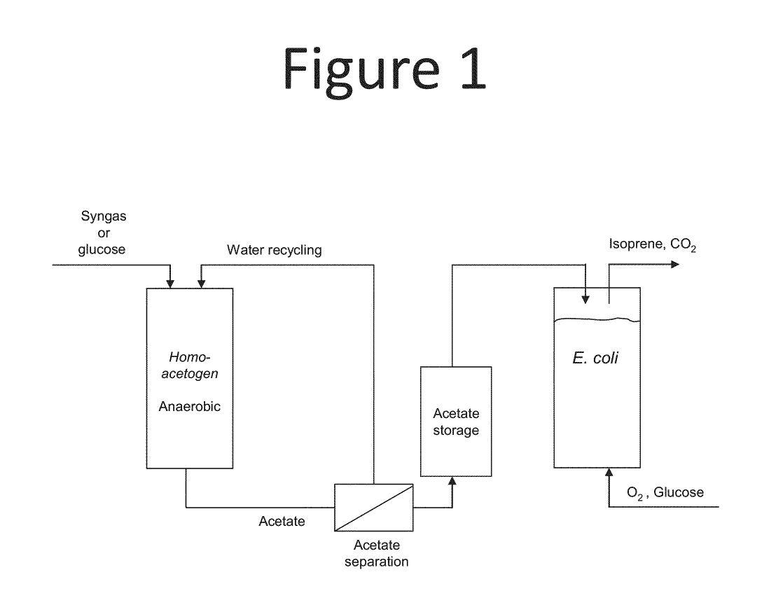

In certain aspects, any of the recombinant cells described herein can be co-cultured in parallel with homoacetogenic bacteria to provide a storable supply of acetate for use as a carbon source in the production of isoprene, isoprenoid, or isoprenoid precursor molecules (e.g., mevalonate (MVA)) (FIG. 1). In one non-limiting example, homoacetogenic bacteria are cultured in a first fermentor containing growth medium under suitable culture conditions for the production of acetate. The growth media in the first fermentor can include, without limitation, synthesis gas or glucose as a carbon source. In some aspects, the growth media is removed from the first fermentor and the acetate produced by the homoacetogenic bacteria separated from the growth media by methods that are well known in the art. See for example, Wood, FASEB, 5:156-163 (1991) and US Pub. App. US 20100273229. In other aspects, the remaining growth media can then be recycled back into the first fermentor. In another aspect, the separated acetate can be stored in a storage tank for later addition to a second fermentor for culturing recombinant isoprene, isoprenoid, or isoprenoid precursor-producing microorganisms by the co-metabolism of glucose and acetate, such as any of those described in any of the methods disclosed herein. In some aspects, the homoacetogenic bacteria are members of the genus Clostridium.

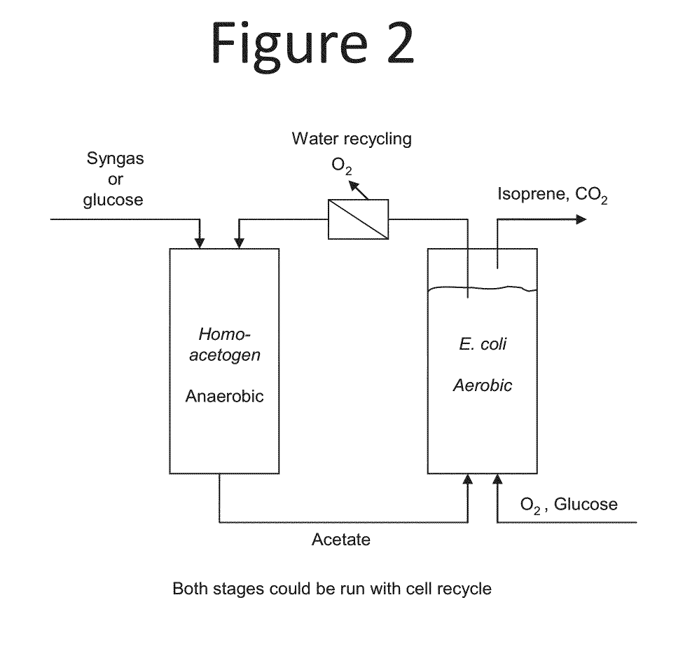

In another non-limiting example, any of the recombinant cells described herein can be co-cultured in parallel with homoacetogenic bacteria to provide a direct source of acetate for the co-metabolism of glucose and acetate by the recombinant cells (FIG. 2). In some aspects, homoacetogenic bacteria are cultured in a first fermentor containing growth medium under suitable culture conditions for the production of acetate. The growth media in the first fermentor can include, without limitation, synthesis gas or glucose as a carbon source. In some aspects, the growth media from the first fermentor containing the homoacetogenic bacteria is directly added to a second fermentor for culturing recombinant isoprene, isoprenoid, or isoprenoid precursor-producing microorganisms, such as any of those described herein. In one aspect, the growth medium from the second fermentor is recycled back into the first fermentor after removal of oxygen from the growth medium by methods that are known in the art. See for example, Wood, FASEB, 5:156-163 (1991) and US Pat. App. Pub. US 20100273229. In some aspects, the homoacetogenic bacteria are members of the genus Clostridium. In other aspects, the recombinant isoprene, isoprenoid, or isoprenoid precursor molecule-producing microorganisms are aerobic microorganisms such as, but not limited to, E. coli.

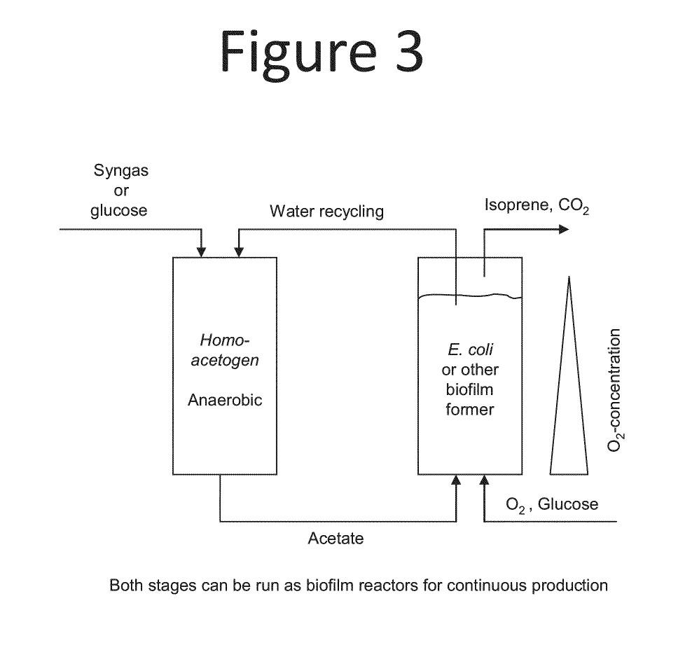

In another non-limiting example, any of the recombinant cells described herein can be co-cultured in parallel with homoacetogenic bacteria to provide a direct source of acetate for the co-metabolism of glucose and acetate by the recombinant cells in an oxygen gradient (FIG. 3). In some aspects, homoacetogenic bacteria are cultured in a first fermentor containing growth medium under suitable culture conditions for the production of acetate. The growth media in the first fermentor can include, without limitation, synthesis gas or glucose as a carbon source. In some aspects, the growth media from the first fermentor containing the homoacetogenic bacteria is directly added to a second fermentor via an inlet located at the bottom of the second fermentor (FIG. 3). The second fermentor is used for culturing recombinant isoprene, isoprenoid, or isoprenoid precursor-producing microorganisms, such as any of those described herein. In one aspect, the second fermentor comprises an oxygen gradient, such that the oxygen concentration at the bottom of the fermentor, near the acetate-containing medium inlet, is greater than the oxygen concentration at the top of the fermentor. In some aspects, the oxygen concentration at the top of the fermentor is about 0%. Media taken from an outlet located on the top of the second fermentor can be recycled back into the first fermentor (FIG. 3). In some aspects, the recombinant isoprene, isoprenoid, or isoprenoid precursor-producing microorganisms are E. coli or other biofilm-forming microorganisms. In other aspects, the homoacetogenic bacteria are members of the genus Clostridium.

### Cell Culture Conditions

Materials and methods suitable for the maintenance and growth of the recombinant cells disclosed herein are described infra, e.g., in the Examples section. Other materials and methods suitable for the maintenance and growth of bacterial cultures are well known in the art. Exemplary techniques may be found in International Publication No. WO 2009/076676, U.S. patent application Ser. No. 12/335,071 (U.S. Publ. No. 2009/0203102), WO 2010/003007, US Publ. No. 2010/0048964, WO 2009/132220, US Publ. No. 2010/0003716, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. In some aspects, the cells are cultured in a culture medium under conditions permitting the expression of one or more isoprene synthase, IDI polypeptides, polyprenyl pyrophosphate synthase polypeptides, MVA pathway polypeptides and/or DXP pathway polypeptides encoded by a nucleic acid inserted into the host cells.

Standard cell culture conditions can be used to culture the cells (see, for example, WO 2004/033646 and references cited therein). In some aspects, cells are grown and maintained at an appropriate temperature, gas mixture, and pH (such as at about 20° C. to about 37° C., at about 6% to about 84% CO₂, and at a pH between about 5 to about 9). In some aspects, cells are grown at 35° C. in an appropriate cell medium. In some aspects, the pH ranges for fermentation are between about pH 5.0 to about pH 9.0 (such as about pH 6.0 to about pH 8.0 or about 6.5 to about 7.0). Reactions may be performed under aerobic, anoxic, or anaerobic conditions based on the requirements of the host cells.

Standard culture conditions and modes of fermentation, such as batch, fed-batch, or continuous fermentation that can be used are described in International Publication No. WO 2009/076676, U.S. patent application Ser. No. 12/335,071 (U.S. Publ. No. 2009/0203102), WO 2010/003007, US Publ. No. 2010/0048964, WO 2009/132220, US Publ. No. 2010/0003716. Batch and Fed-Batch fermentations are common and well known in the art and examples may be found in Brock, Biotechnology: A Textbook of Industrial Microbiology, Second Edition (1989) Sinauer Associates, Inc.

In certain embodiments, culture conditions can comprise pulse feeding glucose to result in periodic (e.g. post-pulse) excess glucose conditions. This can result in excess production of acetate by the cultured cells wherein the excess acetate is released into the culture media. This excess acetate is then re-consumed by the cultured cells. In other embodiments, culture conditions can comprise O₂ limited conditions. In yet other embodiments, the culture conditions can comprise alternately culturing the cells in (i) pulse feeding conditions and (ii) in O₂ limited conditions.

In some aspects, the carbon source includes yeast extract or one or more components of yeast extract. In some aspects, the concentration of yeast extract is 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. In some aspects, the carbon source contains both yeast extract (or one or more components thereof) and other carbon sources, such as glucose and acetate.

In some aspects, the cells are grown in batch culture. In some aspects, the cells are grown in fed-batch culture. In some aspects, the cells are grown in continuous culture. In some aspects, the minimal medium is supplemented with 1.0% (w/v) glucose or less. In some aspects, the minimal medium is supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In certain aspects, the minimal medium is supplemented 0.1% (w/v) or less yeast extract. In some aspects, the minimal medium is supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. In some aspects, the minimal media does not contain yeast extract. In some aspects, the minimal medium is supplemented with 1% (w/v) glucose or less and 0.1% (w/v) or less. In some aspects, the minimal medium is supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. In other aspects, the minimal medium is supplemented with 0.05% (w/v), 0.1% (w/v), 0.2% (w/v), 0.25% (w/v), 0.3% (w/v), 0.4% (w/v), 0.5%, (w/v) 0.6% (w/v), 0.7% (w/v), 0.8% (w/v), 0.9% (w/v), 1.0% (w/v), 1.1% (w/v), 1.2% (w/v), 1.3% (w/v), 1.4% (w/v), or 1.5% (w/v) acetate. In yet other aspects, the minimal medium is supplemented with media having at least about 1% glucose concentration and a concentration of acetate of at least about 0.01% to about 1.5%. In other aspects, the concentration of acetate is at least about 0.01% to about 0.75%, at least about 0.01% to about 0.5%, at least about 0.01% to about 0.4%, at least about 0.01% to about 0.3%, at least about 0.01% to about 0.25%, or at least about 0.01% to about 0.2%. In certain aspects, the minimal medium is supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose, 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract, and/or 0.05% (w/v), 0.1% (w/v), 0.2% (w/v), 0.25% (w/v), 0.3% (w/v), 0.4% (w/v), 0.5%, (w/v) 0.6% (w/v), 0.7% (w/v), 0.8% (w/v), 0.9% (w/v), 1.0% (w/v), 1.1% (w/v), 1.2% (w/v), 1.3% (w/v), 1.4% (w/v), or 1.5% (w/v) acetate.

### Increasing Intracellular Acetyl Co-A

Any of the cells described above can be used in the improved methods for the production of increased intracellular acetyl Co-A disclosed herein. In some aspects, the invention encompasses a method for increasing intracellular acetyl Co-A by a recombinant host cell, the method comprising culturing recombinant host cells in the presence of a carbon source (such as glucose) and acetate under suitable conditions for the production of intracellular acetyl Co-A, wherein the host cells comprise increased expression of pyruvate dehydrogenase and/or malic enzyme; and wherein the intracellular acetyl-CoA concentrations are increased within the recombinant host cells. In some aspects, intracellular acetyl Co-A production by the recombinant host cells cultured in the presence of a carbon source (such as glucose) and acetate is improved compared to the production of intracellular acetyl Co-A by recombinant host cells cultured in the presence of a less oxidized carbon source (for example, but not limited to, glucose) alone.

The cells may additionally comprise one or more heterologous nucleic acid(s) encoding IDI, MVA pathway polypeptides, or DXP pathway polypeptides. In some aspects, heterologous nucleic acid(s) encoding one or more IDI, DXP pathway polypeptides, or MVA pathway polypeptides can be expressed on multicopy plasmids or can be integrated into the chromosome of the host cell. In some aspects, the one or more heterologous nucleic acid(s) encoding IDI, MVA pathway polypeptides, or DXP pathway polypeptides can comprise any number of (such as any of 1, 2, 3, 4, 5, 6, 7, 8, 9, or 10) heterologous nucleic acids. In certain aspects, the heterologous nucleic acid(s) encoding one or more IDI, DXP pathway polypeptides, or MVA pathway polypeptides can be under the control of an inducible promoter or a constitutively expressing promoter. In some aspects, the cells can comprise one or more heterologous nucleic acids encoding polypeptides comprising the entire MVA pathway or at least a component of the MVA pathway (such as upper MVA pathway polypeptides, the lower MVA pathway polypeptides, or a mevalonate kinase polypeptide). In one embodiment, the mevalonate kinase polypeptide can be from the genus Methanosarcina (such as M. mazei). In another embodiment, the mevalonate kinase polypeptide can be from the genus Methanococcoides (such as M. burtonii). In another aspect, any of the heterologous nucleic acids described herein can be expressed on multicopy plasmids or can be integrated into the chromosome of the host cell. Additionally, the recombinant host cells may be deficient in enzymes whose expression is thought to decrease intracellular concentrations of acetyl Co-A. These can include enzymes of the TCA or citric acid cycle (including, but not limited to, citrate synthase) and enzymes involved in lactate metabolism (including, but not limited to, lactate dehydrogenase).

In some aspects of the improved methods for increasing intracellular acetyl Co-A disclosed herein, the cells can be cultured in media having at least about 20% glucose concentration and a concentration of acetate of at least about 0.01% to about 1.5%. The concentration of glucose in the cell culture media may be varied, and can include at least about 20%, 15%, 10%, 7.5%, 5%, 4%, 3%, 2%, 1%, 0.9%, 0.8%, 0.7%, 0.6%, 0.5%, 0.4%, 0.3%, 0.2%, or 0.1% glucose, inclusive, including any percentage value in between these numbers. The concentration of acetate in the cell culture medium may also vary, and can include at least about 0.05%, 0.1%, 0.2%, 0.25%, 0.3%, 0.4%, 0.5%, 0.6%, 0.7%, 0.8%, 0.9%, 1.0%, 1.1%, 1.2%, 1.3%, 1.4%, or 1.5% acetate, inclusive, including any percentage value in between these numbers. In other aspects, the concentration of acetate is at least about 0.01% to about 1.5%, at least about 0.01% to about 1.0%, at least about 0.01% to about 0.75%, at least about 0.01% to about 0.5%, at least about 0.01% to about 0.4%, at least about 0.01% to about 0.3%, at least about 0.01% to about 0.25%, or at least about 0.01% to about 0.2%. In certain other embodiments, the cell culture media can be further supplemented with any of about 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

In some aspects, any of the improved methods for the production of increased intracellular acetyl Co-A disclosed herein can result in increases in intracellular acetyl Co-A concentrations of at least about 10%, 20%, 30%, 40%, 50%, 60%, 70%, 80%, 90%, or 100%, inclusive, including any percentage value in between these numbers, versus cells that are cultured in the presence of a less oxidized carbon source (for example, but not limited to, glucose) alone. In other aspects, at least about one half of the carbon atoms comprising intracellular acetyl Co-A molecules come from acetate when the cells are cultured in the presence of both acetate and glucose versus the source of carbon atoms for intracellular acetyl Co-A when the cells are cultured in the presence of a less oxidized carbon source (for example, but not limited to, glucose) alone.

### Production of Isoprene

Any of the cells described above can be used can be used in the methods for increasing the efficiency, the yield, and the production of isoprene disclosed herein. In some aspects, the invention encompasses a method for improving the efficiency of the production of isoprene by a recombinant host cell, the method comprising culturing recombinant host cells in the presence of a culture media comprising a carbon source and acetate under suitable conditions for the production of isoprene, wherein the host cells comprise one or more heterologous nucleic acids encoding for an isoprene synthase polypeptide; and wherein the recombinant host cells are capable of producing isoprene. In some aspects, isoprene production by the recombinant host cells cultured in the presence of a carbon source (such as glucose) and acetate is improved compared to the isoprene production by recombinant host cells cultured in the presence of a less oxidized carbon source (for example, but not limited to, glucose) alone.

The cells used in any of the methods for improving the efficiency of the production of isoprene disclosed herein may additionally comprise one or more heterologous nucleic acid(s) encoding IDI, MVA pathway polypeptides, or DXP pathway polypeptides. In some aspects, heterologous nucleic acid(s) encoding one or more isoprene synthase, IDI, MVA pathway polypeptides, or DXP pathway polypeptides can be expressed on multicopy plasmids or can be integrated into the chromosome of the host cell. In certain aspects, the heterologous nucleic acid(s) encoding one or more isoprene synthase, IDI, MVA pathway polypeptides or DXP pathway polypeptides can be under the control of an inducible promoter or a constitutively expressing promoter. In some aspects, the one or more heterologous nucleic acid(s) encoding IDI, MVA pathway polypeptides, or DXP pathway polypeptides can comprise any number of (such as any of 1, 2, 3, 4, 5, 6, 7, 8, 9, or 10) heterologous nucleic acids. In some aspects, the cells can comprise one or more heterologous nucleic acids encoding polypeptides comprising the entire MVA pathway or at least a component of the MVA pathway (such as upper MVA pathway polypeptides, the lower MVA pathway polypeptides, or a mevalonate kinase polypeptide). In one embodiment, the mevalonate kinase polypeptide can be from the genus Methanosarcina (such as M. mazei). In another embodiment, the mevalonate kinase polypeptide can be from the genus Methanococcoides (such as M. burtonii).

In some aspects of the methods for improving the efficiency of the production of isoprene disclosed herein, the cells can be cultured in media having at least about 20% glucose concentration and a concentration of acetate of at least about 0.01% to about 1.5%. The concentration of glucose in the cell culture media may be varied, and can include at least about 20%, 15%, 10%, 7.5%, 5%, 4%, 3%, 2%, 1%, 0.9%, 0.8%, 0.7%, 0.6%, 0.5%, 0.4%, 0.3%, 0.2%, or 0.1% glucose, inclusive, including any percentage value in between these numbers. The concentration of acetate in the cell culture medium may also vary, and can include at least about 0.05%, 0.1%, 0.2%, 0.25%, 0.3%, 0.4%, 0.5%, 0.6%, 0.7%, 0.8%, 0.9%, 1.0%, 1.1%, 1.2%, 1.3%, 1.4%, or 1.5% acetate, inclusive, including any percentage value in between these numbers. In other aspects, the concentration of acetate is at least about 0.01% to about 1.5%, is at least about 0.01% to about 1.0%, is at least about 0.01% to about 0.75%, at least about 0.01% to about 0.5%, at least about 0.01% to about 0.4%, at least about 0.01% to about 0.3%, at least about 0.01% to about 0.25%, or at least about 0.01% to about 0.2%. In certain other embodiments, the cell culture media can be further supplemented with any of about 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

In some aspects, the method for improving the efficiency of the production of isoprene is characterized by an increase in the ratio between isoprene and carbon dioxide (CO₂) produced by the cells in culture. This increased ratio of isoprene to carbon dioxide can be found in the fermentation off gas produced by the cultured cells. In certain aspects, the increase in the ratio between isoprene and CO₂ is at least about 5%, 10%, 15%, 20%, 25%, 30%, 35%, 40%, 45%, 50%, 55%, or 65%, inclusive, including any percentages in between these values, when the cells are cultured in the presence of a carbon source (such as glucose) and acetate under suitable conditions for the production of isoprene.

In other aspects, the method for improving the efficiency of the production of isoprene is characterized by an increase in the specific productivity of isoprene by the cells in culture. By “specific productivity,” it is meant absolute amount of isoprene in the off-gas during the culturing of cells for a particular period of time. In some aspects, the improved methods disclosed herein increase the specific productivity of isoprene at least about 10%, 20, 30, 40, 50, 60, 70, 80, 90, or 100%, inclusive, including any percentages in between these values, when the cells are cultured in the presence of a carbon source (such as glucose) and acetate versus the specific productivity of those cells when they are cultured on a less oxidized carbon source (e.g., glucose) alone.

In other aspects, the method for improving the efficiency of the production of isoprene is characterized by an increase in the cumulative yield of isoprene by the cells in culture. By “cumulative yield,” it is meant the absolute amount of isoprene (in grams) produced from the initiation of the fermentation or over a certain period of time (e.g., the last 40 hours) divided by the amount of glucose consumed (in grams) over the same time period (expressed in %). In some aspects, the improved methods disclosed herein increase the cumulative yield of isoprene at least about 1% to about 15%, inclusive, including any percentages in between these values, when the cells are cultured in the presence of a carbon source (such as glucose) and acetate versus the cumulative yield of those cells when they are cultured on a less oxidized carbon source (e.g., glucose) alone.

In other aspects, the method for improving the efficiency of the production of isoprene is characterized by an increase in the Cell Productivity Index (CPI) isoprene by the cells in culture. In some aspects, the improved methods disclosed herein increase the CPI of isoprene at least about 1% to about 15%, inclusive, including any percentages in between these values, when the cells are cultured in the presence of a carbon source (such as glucose) and acetate versus the cumulative yield of those cells when they are cultured on a less oxidized carbon source (e.g., glucose) alone.

In certain aspects of any of the methods disclosed herein, recombinant microorganisms engineered for the production of isoprene and cultured in the presence of both a carbon source (such as glucose) and acetate require less oxygen compared to the same cells cultured in the presence of a less oxidized carbon source (for example, but not limited to, glucose) alone. In some aspects, recombinant cells cultured in the presence of both glucose and acetate require any of about 1%, 2%, 3%, 4%, 5%, 6%, 7, 8%, 9%, 10%, 11%, 12%, 13%, 14%, 15%, 20%, 25%, 30%, 35%, 40%, 45%, or 50% less oxygen, inclusive, including any percentage value in between these numbers.

In other aspects, the recombinant microorganisms engineered for the production of isoprene and cultured in the presence of a carbon source (such as glucose) and acetate produce less carbon dioxide compared to the same cells cultured in the presence of a less oxidized carbon source (for example, but not limited to, glucose) alone. Less carbon dioxide evolution by cultured microorganisms during the isoprene production process is environmentally advantageous, as it reduces the greenhouse gas emissions associated with large-scale fermentations. In some aspects, recombinant cells cultured in the presence of both a carbon source (such as glucose) and acetate produce any of about 1%, 2%, 3%, 4%, 5%, 6%, 7, 8%, 9%, 10%, 11%, 12%, 13%, 14%, 15%, 20%, 25%, 30%, 35%, 40%, or 45% less carbon dioxide, inclusive, including any percentage value in between these numbers.

Engineering Recombinant Cells for Production of Isoprenoid and/or Isoprenoid Precursor Molecules

Isoprenoids are produced by many organisms from the synthesis of isoprenoid precursor molecules which are the end products of the MVA and DXP biosynthetic pathways. As stated above, isoprenoids represent an important class of compounds and include, for example, food and feed supplements, flavor and odor compounds, and anticancer, antimalarial, antifungal, and antibacterial compounds.

Microorganisms can be engineered to produce isoprenoids and/or isoprenoid precursor molecules. As a class of molecules, isoprenoids are classified based on the number of isoprene units present in the compound. Monoterpenes comprise ten carbons or two isoprene units, sesquiterpenes comprise 15 carbons or three isoprene units, diterpenes comprise 20 carbons or four isoprene units, sesterterpenes comprise 25 carbons or five isoprene units, and so forth. Steroids (generally comprising about 27 carbons) are the products of cleaved or rearranged isoprenoids.

Isoprenoids can be produced from the isoprenoid precursor molecules IPP and DMAPP. The structurally diverse class of isoprenoid compounds are all derived from these rather simple universal precursors and are synthesized by groups of conserved polyprenyl pyrophosphate synthases (Hsieh et al., Plant Physiol. 2011 March; 155(3):1079-90). The various chain lengths of these linear prenyl pyrophosphates, reflecting their distinctive physiological functions, in general are determined by the highly developed active sites of polyprenyl pyrophosphate synthases via condensation reactions of allylic substrates (dimethylallyl diphosphate (C₅-DMAPP), geranyl pyrophosphate (C₁₀-GPP), farnesyl pyrophosphate (C₁₅-FPP), geranylgeranyl pyrophosphate (C₂₀-GGPP)) with a corresponding number of isopentenyl pyrophosphates (C₅-IPP) (Hsieh et al., Plant Physiol. 2011 March; 155(3):1079-90).

Isoprenoid precursors and/or isoprenoids can be produced using any of the recombinant host cells described herein. In some aspects, these cells further comprise one or more heterologous nucleic acids encoding polypeptides of the MVA pathway, IDI, and/or the DXP pathway, as described above, and a heterologous nucleic acid encoding a polyprenyl pyrophosphate synthase polypeptide.

Types of Isoprenoids

The recombinant cells of the present invention are capable of increased production of isoprenoids and the isoprenoid precursor molecules DMAPP and IPP. Examples of isoprenoids include, without limitation, hemiterpenoids, monoterpenoids, sesquiterpenoids, diterpenoids, sesterterpenoids, triterpenoids, tetraterpenoids, and higher polyterpenoids. In some aspects, the hemiterpenoid is prenol (i.e., 3-methyl-2-buten-1-ol), isoprenol (i.e., 3-methyl-3-buten-1-ol), 2-methyl-3-buten-2-ol, or isovaleric acid. In some aspects, the monoterpenoid can be, without limitation, geranyl pyrophosphate, eucalyptol, limonene, or pinene. In some aspects, the sesquiterpenoid is farnesyl pyrophosphate, artemisinin, or bisabolol. In some aspects, the diterpenoid can be, without limitation, geranylgeranyl pyrophosphate, retinol, retinal, phytol, taxol, forskolin, or aphidicolin. In some aspects, the triterpenoid can be, without limitation, squalene or lanosterol. The isoprenoid can also be selected from the group consisting of abietadiene, amorphadiene, carene, α-farnesene, β-farnesene, farnesol, geraniol, geranylgeraniol, linalool, limonene, myrcene, nerolidol, ocimene, patchoulol, β-pinene, sabinene, γ-terpinene, terpindene and valencene.

In some aspects, the tetraterpenoid is lycopene or carotene (a carotenoid). As used herein, the term “carotenoid” refers to a group of naturally-occurring organic pigments produced in the chloroplasts and chromoplasts of plants, of some other photosynthetic organisms, such as algae, in some types of fungus, and in some bacteria. Carotenoids include the oxygen-containing xanthophylls and the non-oxygen-containing carotenes. In some aspects, the carotenoids are selected from the group consisting of xanthophylls and carotenes. In some aspects, the xanthophyll is lutein or zeaxanthin. In some aspects, the carotenoid is α-carotene, β-carotene, γ-carotene, β-cryptoxanthin or lycopene.

Polyprenyl Pyrophosphate Synthases Polypeptides and Nucleic Acids

In some aspects of the invention, the cells described in any of the methods disclosed herein further comprise one or more nucleic acids encoding a polyprenyl pyrophosphate synthase polypeptide(s). The polyprenyl pyrophosphate synthase polypeptide can be an endogenous polypeptide. The endogenous nucleic acid encoding a polyprenyl pyrophosphate synthase polypeptide can be operably linked to a constitutive promoter or can similarly be operably linked to an inducible promoter. In particular, the cells can be engineered to over-express the endogenous polyprenyl pyrophosphate synthase polypeptide relative to wild-type cells.

In some aspects, the polyprenyl pyrophosphate synthase polypeptide is a heterologous polypeptide. The cells of the present invention can comprise more than one copy of a heterologous nucleic acid encoding a polyprenyl pyrophosphate synthase polypeptide. In some aspects, the heterologous nucleic acid encoding a polyprenyl pyrophosphate synthase polypeptide is operably linked to a constitutive promoter. In some aspects, the heterologous nucleic acid encoding a polyprenyl pyrophosphate synthase polypeptide is operably linked to an inducible promoter.

The nucleic acids encoding a polyprenyl pyrophosphate synthase polypeptide(s) can be integrated into a genome of the host cells or can be stably expressed in the cells. The nucleic acids encoding a polyprenyl pyrophosphate synthase polypeptide(s) can additionally be on a vector.

Exemplary polyprenyl pyrophosphate synthase nucleic acids include nucleic acids that encode a polypeptide, fragment of a polypeptide, peptide, or fusion polypeptide that has at least one activity of a polyprenyl pyrophosphate synthase. Polyprenyl pyrophosphate synthase polypeptides convert isoprenoid precursor molecules into more complex isoprenoid compounds. Exemplary polyprenyl pyrophosphate synthase polypeptides include polypeptides, fragments of polypeptides, peptides, and fusions polypeptides that have at least one activity of an isoprene synthase polypeptide. Exemplary polyprenyl pyrophosphate synthase polypeptides and nucleic acids include naturally-occurring polypeptides and nucleic acids from any of the source organisms described herein. In addition, variants of polyprenyl pyrophosphate synthase can possess improved activity such as improved enzymatic activity. In some aspects, a polyprenyl pyrophosphate synthase variant has other improved properties, such as improved stability (e.g., thermo-stability), and/or improved solubility. Exemplary polyprenyl pyrophosphate synthase nucleic acids can include nucleic acids which encode polyprenyl pyrophosphate synthase polypeptides such as, without limitation, geranyl diphosphosphate (GPP) synthase, farnesyl pyrophosphate (FPP) synthase, and geranylgeranyl pyrophosphate (GGPP) synthase, or any other known polyprenyl pyrophosphate synthase polypeptide.

In some aspects of the invention, the cells described in any of the methods disclosed herein further comprise one or more nucleic acids encoding a farnesyl pyrophosphate (FPP) synthase. The FPP synthase polypeptide can be an endogenous polypeptide encoded by an endogenous gene. In some aspects, the FPP synthase polypeptide is encoded by an endogenous ispA gene in E. coli. The endogenous nucleic acid encoding an FPP synthase polypeptide can be operably linked to a constitutive promoter or can similarly be operably linked to an inducible promoter. In particular, the cells can be engineered to over-express the endogenous FPP synthase polypeptide relative to wild-type cells.

In some aspects, the FPP synthase polypeptide is a heterologous polypeptide. The cells of the present invention can comprise more than one copy of a heterologous nucleic acid encoding a FPP synthase polypeptide. In some aspects, the heterologous nucleic acid encoding a FPP synthase polypeptide is operably linked to a constitutive promoter. In some aspects, the heterologous nucleic acid encoding a FPP synthase polypeptide is operably linked to an inducible promoter. In some aspects, the heterologous nucleic acid encoding a polyprenyl pyrophosphate synthase polypeptide is operably linked to a strong promoter.

The nucleic acids encoding an FPP synthase polypeptide can be integrated into a chromosome of the host cells or can be stably expressed in the cells. The nucleic acids encoding an FPP synthase can additionally be on a vector.

Standard methods can be used to determine whether a polypeptide has polyprenyl pyrophosphate synthase polypeptide activity by measuring the ability of the polypeptide to convert IPP into higher order isoprenoids in vitro, in a cell extract, or in vivo. These methods are well known in the art and are described, for example, in U.S. Pat. No. 7,915,026; Hsieh et al., Plant Physiol. 2011 March; 155(3):1079-90; Keeling et al., BMC Plant Biol. 2011 Mar. 7; 11:43; Martin et al., BMC Plant Biol. 2010 Oct. 21; 10:226; Kumeta & Ito, Plant Physiol. 2010 December; 154(4):1998-2007; and Köllner & Boland, J Org Chem. 2010 Aug. 20; 75(16):5590-600.

Production of Isoprenoids and/or Isoprenoid Precursor Molecules

Any of the cells described above can be used in the methods for improving the efficiency, yield, and/or the production of isoprenoids and/or isoprenoid precursor molecules disclosed herein. In some aspects, the invention encompasses a method for improving the efficiency of the production of isoprenoids and/or isoprenoid precursor molecules by a recombinant host cell, the method comprising culturing recombinant host cells in the presence of a carbon source (such as glucose) and acetate under suitable conditions for the production of isoprenoids and/or isoprenoid precursor molecules, wherein the host cells comprise one or more heterologous nucleic acids encoding for a polyprenyl pyrophosphate synthase polypeptide; and wherein the recombinant host cells are capable of producing isoprenoids and/or isoprenoid precursor molecules. In some aspects, the efficiency of isoprenoid and/or isoprenoid precursor molecule production by the recombinant host cells cultured in the presence of a carbon source (such as glucose) and acetate is improved compared to the production of these compounds by recombinant host cells cultured in the presence of a less oxidized carbon source (for example, but not limited to, glucose) alone.

The cells may additionally comprise one or more heterologous nucleic acid(s) encoding IDI, MVA pathway polypeptides, or DXP pathway polypeptides. In some aspects, heterologous nucleic acid(s) encoding one or more polyprenyl pyrophosphate synthase, IDI, DXP pathway polypeptides, or MVA pathway polypeptides can be expressed on multicopy plasmids or can be integrated into the chromosome of the host cell. In some aspects, the one or more heterologous nucleic acid(s) encoding IDI, MVA pathway polypeptides, or DXP pathway polypeptides can comprise any number of (such as any of 1, 2, 3, 4, 5, 6, 7, 8, 9, or 10) heterologous nucleic acids. In certain aspects, the heterologous nucleic acid(s) encoding one or more polyprenyl pyrophosphate synthase, IDI, DXP pathway polypeptides, or MVA pathway polypeptides can be under the control of an inducible promoter or a constitutively expressing promoter. In some aspects, the cells can comprise one or more heterologous nucleic acids encoding polypeptides comprising the entire MVA pathway or at least a component of the MVA pathway (such as upper MVA pathway polypeptides, the lower MVA pathway polypeptides, or a mevalonate kinase polypeptide). In one embodiment, the mevalonate kinase polypeptide can be from the genus Methanosarcina (such as M. mazei). In another embodiment, the mevalonate kinase polypeptide can be from the genus Methanococcoides (such as M. burtonii).

In some aspects of the methods for improving the efficiency of the production of isoprenoids and/or isoprenoid precursor molecules disclosed herein, the cells can be cultured in media having at least about 20% glucose concentration and a concentration of acetate of at least about 0.01% to about 1.5%. The concentration of glucose in the cell culture media may be varied, and can include at least about 20%, 15%, 10%, 7.5%, 5%, 4%, 3%, 2%, 1%, 0.9%, 0.8%, 0.7%, 0.6%, 0.5%, 0.4%, 0.3%, 0.2%, or 0.1% glucose, inclusive, including any percentage value in between these numbers. The concentration of acetate in the cell culture medium may also vary, and can include at least about 0.05%, 0.1%, 0.2%, 0.25%, 0.3%, 0.4%, 0.5%, 0.6%, 0.7%, 0.8%, 0.9%, 1.0%, 1.1%, 1.2%, 1.3%, 1.4%, or 1.5% acetate, inclusive, including any percentage value in between these numbers. In other aspects, the concentration of acetate is at least about 0.01% to about 1.5%, at least about 0.01% to about 1.0%, at least about 0.01% to about 0.75%, at least about 0.01% to about 0.5%, at least about 0.01% to about 0.4%, at least about 0.01% to about 0.3%, at least about 0.01% to about 0.25%, or at least about 0.01% to about 0.2%. In certain other embodiments, the cell culture media can be further supplemented with any of about 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

In other aspects, the methods for improving the efficiency of the production of isoprenoids and/or isoprenoid precursor molecules (e.g., mevalonate (MVA)) are characterized by an increase in the specific productivity of isoprenoids and/or isoprenoid precursor molecules (e.g., mevalonate (MVA)) by the cells in culture. By “specific productivity,” it is meant absolute amount of isoprenoids and/or isoprenoid precursor molecules (e.g., mevalonate (MVA)) in the off-gas during the culturing of cells for a particular period of time. In some aspects, the improved methods disclosed herein increase the specific productivity of isoprenoids and/or isoprenoid precursor molecules (e.g., mevalonate (MVA)) at least about 10%, 20, 30, 40, 50, 60, 70, 80, 90, or 100%, inclusive, including any percentages in between these values, when the cells are cultured in the presence of a carbon source (such as glucose) and acetate versus the specific productivity of those cells when they are cultured on a less oxidized carbon source (for example, but not limited to, glucose) alone.

In some aspects of the methods for improving the efficiency of the production of isoprenoids disclosed herein, the isoprenoids produced can be classified as a terpenoid or a carotenoid. In other aspects, the isoprenoid can be classified as any of a monoterpene, a diterpene, a triterpene, a tetraterpene, a sesquiterpene, or a polyterpene. More specifically, the isoprenoids produced by the cells in culture can be any of abietadiene, amorphadiene, carene, α-farnesene, β-farnesene, farnesol, geraniol, geranylgeraniol, linalool, limonene, myrcene, nerolidol, ocimene, patchoulol, β-pinene, sabinene, γ-terpinene, terpindene, or valencene.

In certain aspects of any of the methods disclosed herein, recombinant microorganisms engineered for the production of isoprenoids and/or isoprenoid precursor molecules (e.g., mevalonate (MVA)) and cultured in the presence of both a carbon source (such as glucose) and acetate require less oxygen compared to the same cells cultured in the presence of a less oxidized carbon source (for example, but not limited to, glucose) alone. In some aspects, recombinant cells cultured in the presence of both glucose and acetate require any of about 1%, 2%, 3%, 4%, 5%, 6%, 7, 8%, 9%, 10%, 11%, 12%, 13%, 14%, 15%, 20%, 25%, 30%, 35%, 40%, 45%, or 50% less oxygen, inclusive, including any percentage value in between these numbers. In other aspects, the recombinant microorganisms engineered for the production of isoprenoids and/or isoprenoid precursor molecules (e.g., mevalonate (MVA)) and cultured in the presence of both glucose and acetate produce less carbon dioxide compared to the same cells cultured in the presence of a less oxidized carbon source (for example, but not limited to, glucose) alone. In some aspects, recombinant cells cultured in the presence of both glucose and acetate produce any of about 1%, 2%, 3%, 4%, 5%, 6%, 7, 8%, 9%, 10%, 11%, 12%, 13%, 14%, 15%, 20%, 25%, 30%, 35%, 40%, or 45% less carbon dioxide, inclusive, including any percentage value in between these numbers.

### Exemplary Purification Methods

In some aspects, any of the methods described herein further include a step of recovering the isoprene, isoprenoids, and isoprenoid precursor compounds produced. Additionally, in some aspects, any of the methods described herein can further include a step of recovering isoprene. In some aspects, the isoprene is recovered by absorption stripping (See, e.g., U.S. Patent Appl. Pub. No. 2011/017826 A1). In some aspects, any of the methods described herein further include a step of recovering terpenoid or carotenoid.

Suitable purification methods are described in more detail in U.S. Patent Application Publication No. 2010/0196977 A1.

The invention can be further understood by reference to the following examples, which are provided by way of illustration and are not meant to be limiting.

### EXAMPLES

### Experiment 1

### Use of Glucose and Acetate to Increase Isoprene Yield in Cultured Microorganisms

The purpose of this experiment was to show that glucose and acetate co-metabolism can increase the yield of isoprene production.

### Materials and Methods

Media Recipe (Per Liter Fermentation Media):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 1 g, 1000× Trace Metal Solution 1 ml. All of the components were added together and dissolved in diH₂O. The pH was adjusted to 6.8 with ammonium hydroxide (30%) and brought to volume. Media was filter sterilized with a 0.22 micron vacuum filter. Antibiotics were added after sterilization and pH adjustment. The media contained 0.02% yeast extract. Glucose was added to the media to a final concentration of 1%. Acetate was added to a concentration ranging from 0 to 1% during the experiment.

1000× Trace Metal Solution (Per Liter Fermentation Media):

Citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO4*2H₂O 100 mg. Each component was dissolved one at a time in diH₂O, pH to 3.0 with HCl/NaOH, and then brought to volume and filter sterilized with 0.22 micron filter.

Strains:

MD09-317: BL21 (DE3), [t pgl FRT-PL.2-mKKDyl, pCLUpper (pMCM82) (Spec50), pTrcAlba(MEA)mMVK (pDW34) (Carb50)] containing the upper mevalonic acid pathway (pCL Upper) and the lower MVA pathway including isoprene synthase from Alba (pTrcAlba(MRA)mMVK). MD09-317 was constructed by transducing the lower pathway gene PL.2-mKKDyI in CMP258 (BL21 wt+pgl) host strain, using a lysate PL.2-mKKDyI::KanR made from the MCM521 host strain. The resulting construct was named MD09-313. The resistance Kan marker was subsequently removed. The resulting strain was called HMB=BL21 wt, pgl+t PL.2-mKKDyI::FRT. Once the marker is removed, pathway plasmids (MCM82 & pDW34) were transformed in an HMB host to create MD09-317.

Experimental Procedure:

The isoprene producing strain MD09-317 containing the MVA pathway and isoprene synthase was grown from a single colony overnight and diluted to an OD of 0.05 in fresh Tm3 media with 1% glucose and 0.02% yeast extract. Cells were induced with either 100 uM IPTG from the beginning of the experiment. The cells were grown in a volume of 4.5 mL using a 24-well Microreactor (MicroReactor Techonologies, Inc., Mountain View, Calif.) at 34° C. to an optical density of approximately 1.2 (measured at 550 nm in a 1 cm cuvette). Sodium acetate was then added to a final concentration of either 0%, 0.05%, 0.1%, 0.25%, 0.5% or 1%. All conditions were run in duplicate. The off-gas from the bioreactors was analyzed using an on-line Hiden HPR-20 mass spectrometer. Masses corresponding to isoprene, CO₂ and other gasses naturally occurring in air were monitored. Concentration of isoprene and CO₂ was calculated as percentage of the off-gas.

### Results

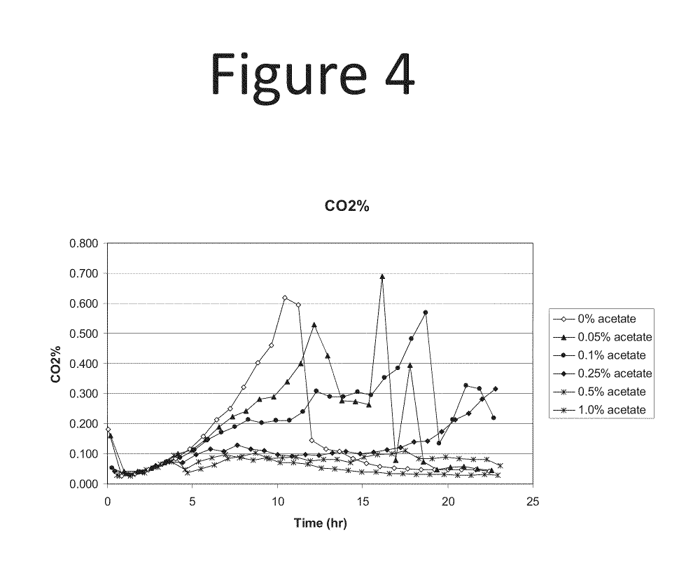

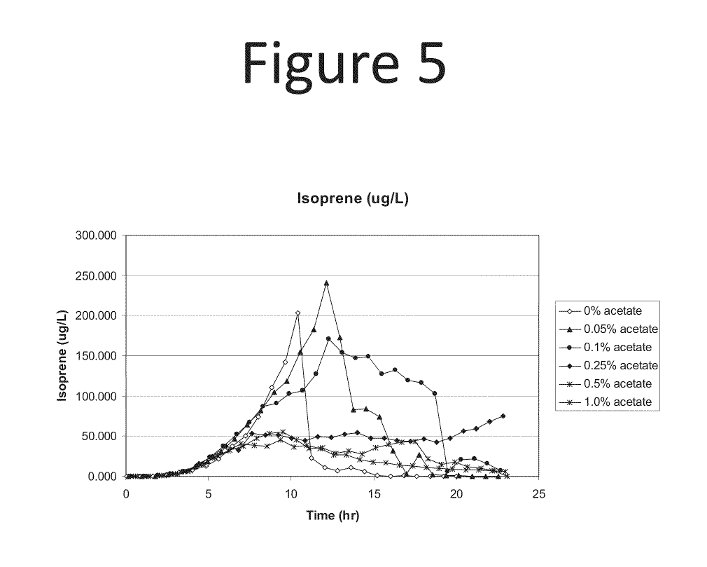

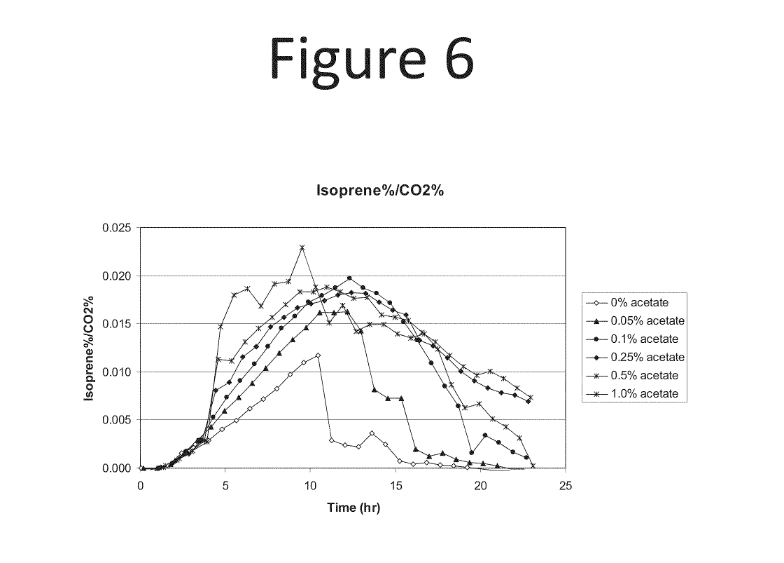

The isoprene producing strain MD09-317 was grown to an optical density of approximately 1.2 in media containing 1% glucose before acetate was added at concentrations ranging from 0% to 1% during exponential growth. The addition of acetate at all concentrations resulted in a decrease in respiration (FIG. 4). A significant reduction in growth and respiration was observed for acetate concentrations of 0.25% and higher. The amount of isoprene emitted from the microreactors showed only a slight decrease at acetate concentrations up to 0.1% (FIG. 5). At higher concentrations, the total isoprene production was significantly lower due to the decreased respiration and accumulation of biomass. The amount of isoprene in the off-gas (% isoprene) divided with the amount of CO₂ (% CO₂) can be used as a measure of the yield of the isoprene forming reaction. Surprisingly, the ratio between isoprene % and CO₂% in the off-gas increased significantly after the addition of acetate to the cultures (FIG. 6). Moderate concentrations of acetate were found to slow down growth of the cells while maintaining high isoprene production for extended periods of time. For example, addition of 0.1% acetate resulted in significantly lower CO₂ emission, when compared to the glucose control that continued to grow exponentially. However, isoprene emission at 0.1% acetate was comparable to the glucose control. The decreased growth and CO₂ emission in the presence of 0.1% acetate resulted in approximately 60% increase in Isoprene %/CO₂%. Higher concentrations of acetate resulted in ever larger increases in yield, but were also found to limit the total isoprene production due to slow growth/metabolism. These data demonstrate that glucose and acetate co-metabolism has the potential of significantly increasing the yield of isoprene formation.

### Example 2

### Acetate Conversion into Isoprene by E. Coli

The purpose of this experiment was to demonstrate that acetate can be taken up by E. coli while growing on glucose and that the acetate can be converted into isoprene via acetyl-Co-A. The experiment proves that glucose and acetate can be co-metabolized and converted into isoprene.

### Materials and Methods

Media Recipe (Per Liter Fermentation Media):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 1 g, 1000× Trace Metal Solution 1 ml. All of the components were added together and dissolved in diH₂O. The pH was adjusted to 6.8 with ammonium hydroxide (30%) and brought to volume. Media was filter sterilized with a 0.22 micron vacuum filter. Antibiotics were added after sterilization and pH adjustment. The media contained 0.02% yeast extract. Fully ¹³C-labeled glucose was added to the media to a final concentration of 1%. Fully ¹²C-labeled acetate was added to a concentration ranging from 0.1% to 0.5% during exponential growth.

1000× Trace Metal Solution (Per Liter Fermentation Media):

Citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO₄*2H₂O 100 mg. Each component is dissolved one at a time in diH₂O, pH to 3.0 with HCl/NaOH, and then brought to volume and filter sterilized with 0.22 micron filter.

Strains:

MD09-317: BL21 (DE3), [t pgl FRT-PL.2-mKKDyl, pCLUpper (pMCM82) (Spec50), pTrcAlba(MEA)mMVK (pDW34) (Carb50)] containing the upper mevalonic acid pathway (pCL Upper) and the lower MVA pathway including isoprene synthase from Alba (pTrcAlba(MRA)mMVK). MD09-317 was constructed by transducing the lower pathway gene PL.2-mKKDyI in CMP258 (BL21 wt+pgl) host strain, using a lysate PL.2-mKKDyI::KanR made from the MCM521 host strain. The resulting construct was named MD09-313. The resistance Kan marker was subsequently removed. The resulting strain was called HMB=BL21 wt, pgl+t PL.2-mKKDyI::FRT. Once the marker is removed, pathway plasmids (MCM82 & pDW34) were transformed in an HMB host to create MD09-317.

Experimental Procedure:

The isoprene producing strain MD09-317 containing the MVA pathway and isoprene synthase was grown from a single colony overnight and diluted to an OD of 0.05 in fresh Tm3 media with 1% fully ¹³C-labeled glucose and 0.02% yeast extract. Cells were induced with either 100 or 200 μM IPTG from the beginning of the experiment. The cells were grown in a volume of 4.5 mL using a 24-well Microreactor (MicroReactor Techonologies, Inc., Mountain View, Calif.) at 34° C. to an optical density of approximately 1.2 (measured at 550 nm in a 1 cm cuvette). Fully unlabeled sodium acetate was then added to a final concentration of either 0.1%, 0.25%, or 0.5%. All conditions were run in duplicate. The off-gas from the bioreactors was analyzed using an on-line Hiden HPR-20 mass spectrometer. Masses corresponding to the different isotopomers of isoprene, CO₂ and other gasses naturally occurring in air were monitored. Concentrations of isoprene and CO₂ were calculated as percentage of the off-gas. The distribution of labeled (¹³C from glucose) versus unlabeled (¹²C from acetate) carbon in the acetyl group in the intracellular pool of acetyl-Co-A was derived from the concentration of the different isotopomers of isoprene in the off-gas.

### Results

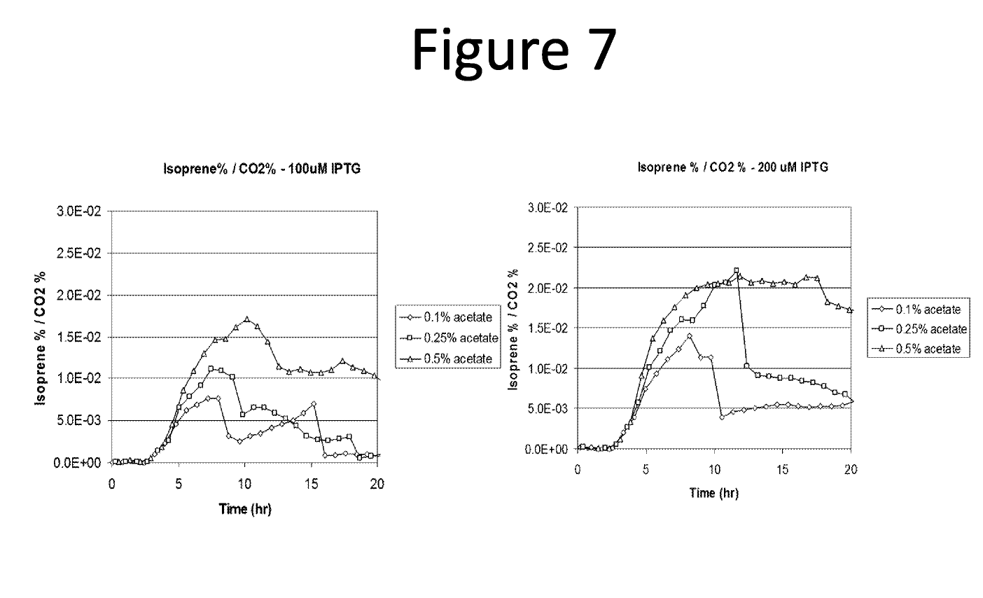

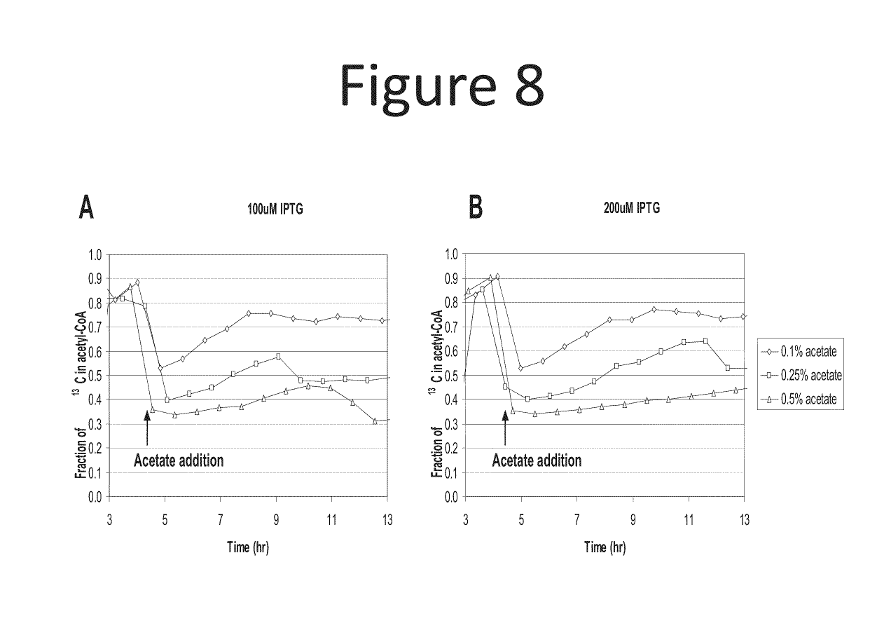

Addition of acetate to a culture growing on glucose showed an increase in the ratio between Isoprene and CO₂ in the off-gas (FIGS. 6 and 7). Bacteria grown on fully ¹³C labeled glucose produced isoprene mostly labeled with ¹³C, indicating that most of the carbon came from glucose in the media. The fraction of ¹³C labeling in the acetyl group of acetyl-Co-A, a precursor for the MVA pathway, was calculated and was found to be close to 0.8-0.9 during growth on fully ¹³C-labeled glucose (FIG. 8). The addition of ¹²C labeled acetate resulted in a significant change in the fraction of ¹³C-labeled isoprene produced by the strain. After addition of ¹²C-labeled acetate, the fraction of ¹³C in the acetyl-group of acetyl-Co-A dropped to between 0.4 and 0.55, indicating that about half of the carbon that was converted into acetyl-Co-A (and further into isoprene) came from acetate. This proves, surprisingly, that the E. coli can co-metabolize glucose and acetate and simultaneously convert both compounds into isoprene.

### Example 3

### Use of Acetate to Increase Intracellular Acetyl-Co-A Concentration and Specific Productivity of Isoprene

The purpose of this experiment was to demonstrate that addition of acetate to an E. coli culture growing on glucose results in an increase in the intracellular concentration of acetyl-Co-A and to demonstrate that the addition of acetate increases the specific productivity of isoprene.

### Materials and Methods

Media Recipe (Per Liter Fermentation Media):

K₂HPO₄ 13.6 g, KH₂PO₄ 13.6 g, MgSO₄*7H₂O 2 g, citric acid monohydrate 2 g, ferric ammonium citrate 0.3 g, (NH₄)₂SO₄ 3.2 g, yeast extract 1 g, 1000× Trace Metal Solution 1 ml. All of the components were added together and dissolved in diH₂O. The pH was adjusted to 6.8 with ammonium hydroxide (30%) and brought to volume. Media was filter sterilized with a 0.22 micron vacuum filter. Antibiotics were added after sterilization and pH adjustment. The media contained 0.02% yeast extract. Glucose was added to the media to a final concentration of 0.5%. Sodium-acetate was added to a concentration ranging from 0% to 0.5% during exponential growth.

1000× Trace Metal Solution (Per Liter Fermentation Media):

Citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO₄*2H₂O 100 mg. Each component is dissolved one at a time in diH₂O, pH to 3.0 with HCl/NaOH, and then brought to volume and filter sterilized with 0.22 micron filter.

Strains:

EWL256: BL21 (DE3), pCLupper (Spec 50), cmR-gi1.2yKKDyl, pTrcAlba-mMVK (carb50) containing the upper mevalonic acid pathway (pCL Upper) and the lower MVA pathway including isoprene synthase from P. Alba was constructed. EWL251 cells were grown in LB to midlog phase and then washed three times in ice-cold, sterile water. Mixed 50 μl of cell suspension with 1 μl of plasmid MCM82 (which is pCL PtrcUpperPathway encoding E. faecalis mvaE and mvaS). The cell suspension mixture was electroporated in a 2 mm cuvette at 2.5 KiloVolts and 25 uFd using a Gene Pulser Electroporator. 1 ml of LB was immediately added to the cells, then transferred to a 14 ml polypropylene tube with a metal cap. Cells were allowed to recover by growing for 2 hour at 30° C. Transformants were selected on LA and 50 μg/μl carbenicillin and 50 μg/μl spectinomycin plates and incubated at 37° C. Picked one colony and designated as strain EWL256.

Experimental Procedure:

The isoprene producing strain EWL256 containing the MVA pathway and isoprene synthase was grown from a single colony overnight and diluted to an OD of 0.05 in fresh Tm3 media with 0.5% glucose and 0.02% yeast extract. The cultures were induced with 200 μM IPTG from the beginning of the experiment.

The cells were grown in a volume of 20 mL in conical flasks at 30° C. to an optical density of approximately 0.7 (measured at 550 nm in a 1 cm cuvette). Sodium acetate was then added to a final concentration of either 0%, 0.05%, 0.25% or 0.5%. All conditions were run in duplicate. After 40 minutes of incubation, 100 μL samples were transferred to 2 mL vials and the amount of isoprene produced in 30 min was determined by GC-MS. The specific isoprene productivity was calculated from these data. Additionally, 1.5 mL of sample was spun down and quenched in 50% methanol at −70° C. The intracellular concentration of acetyl-Co-A was determined using LC-MS.

### Results

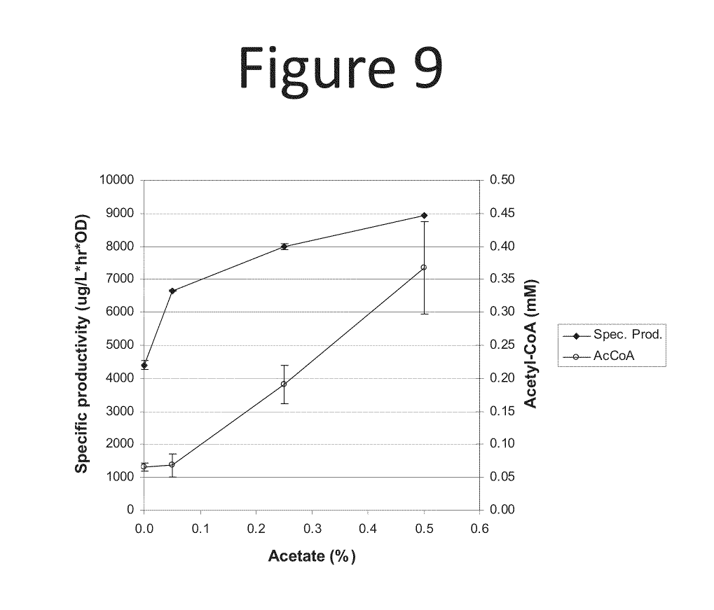

Addition of acetate during exponential growth on glucose resulted in a significant increase in the specific isoprene productivity (FIG. 9). The addition of 0.05% acetate resulted in a nearly 60% increase in the specific isoprene productivity. At higher concentrations of acetate (0.5%), a 100% increase in specific productivity was demonstrated. The intracellular concentration of acetyl-Co-A was also found to increase with the addition of acetate (FIG. 9). With the addition of 0.25% acetate, the intracellular concentration of acetyl-Co-A was found to increase from 0.06 mM to 0.19 mM. From other experiments and from modeling of the pathways, an increase in acetyl-Co-A concentration has been shown to increase the flux through the MVA pathway. Surprisingly. it is therefore demonstrated that the co-metabolism of glucose and acetate can increase the intracellular concentration of acetyl-Co-A and also increase the specific isoprene productivity of cells in culture.

### Experiment 4

### Use of Glucose and Acetate to Increase MVA Yield in Cultured Microorganisms

The purpose of this experiment was to show that glucose and acetate co-metabolism can increase the yield of MVA production.

### Materials and Methods

Media Recipe (Per Liter Fermentation Media):

K₂HPO₄ 13.6 g, KH₂PO₄ 13.6 g, citric acid monohydrate 2 g, ferric ammonium citrate 0.3 g, (NH₄)₂SO₄ 3.2 g, Trace Metal Solution 1 ml. All of the components were added together and dissolved in diH₂O. The pH was adjusted to 6.8 with ammonium hydroxide (30%) and brought to a final volume of 1 L. Media was sterilized by autoclaving and supplemented with 8 mL of 1M filter-sterilized solution of MgSO₄, appropriate antibiotics, 20 mL of 50% filter-sterilized glucose solution, and 0.2 mL of 10% filter-sterilized yeast extract solution. Sodium acetate was added to a final concentration ranging from 0 to 1.8% during the experiment.

1000× Trace Metal Solution (Per Liter Fermentation Media):

Citric Acids*H₂O 40 g, MnSO₄*H₂O 30 g, NaCl 10 g, FeSO₄*7H₂O 1 g, CoCl₂*6H₂O 1 g, ZnSO₄*7H₂O 1 g, CuSO₄*5H₂O 100 mg, H₃BO₃ 100 mg, NaMoO4*2H₂O 100 mg. Each component was dissolved one at a time in diH₂O, pH to 3.0 with HCl/NaOH, and then brought to volume and filter sterilized with 0.22 micron filter.

Construction of Strain CHL936:

Strain CMP1133 (BL21 Δpgl PL.2mKKDyI GI1.2gltA yhfSFRTPyddVIspAyhfS thiFRTtruncIspA) was modified such that the portion of the chromosome containing the lower mevalonic acid pathway genes was deleted to yield strain MD12-778. MD12-778 was electroporated with a plasmid harboring the upper mevalonic acid pathway genes from E. gallinarum (pMCM1225) to yield strain CHL936. Strain CHL936, contains the upper MVA pathway genes from E. gallinarum.

Experimental Procedure:

The strain CHL936 containing the upper MVA pathway was grown from a single colony overnight and diluted to an OD of 0.05 with fresh media (final volume of 100 mL; OD measurements were done at 600 nm in a 1 cm cuvette). After 2.8 hrs of growth in a shake flask at 34° C., cells were induced with 100 uM IPTG, incubated until the culture reached OD of 0.33 and then split into four 20-mL subcultures that were supplemented with sodium acetate to a final concentration of either 0, 0.045, 0.090, or 0.18% (w/v). The cultures were incubated for additional 3.5 hrs upon shaking at 34° C. and the concentrations of glucose, acetate and MVA in the media at the beginning and at the end of the incubation period were analyzed by HPLC (acetate and MVA) or GC (glucose).

HPLC Information:

System: Waters Alliance 2695. Column: BioRad-Aminex HPX-87H Ion Exclusion Column 300 mm×7.8 mm Catalog #125-0140. Column Temperature: 50° C. Guard column: BioRad-Microguard Cation H refill 30 mm×4.6 mm Catalog #125-0129. Running buffer: 0.01N H₂SO₄. Running buffer flow rate: 0.6 mL/min. Approximate running pressure: ˜1100-1200 psi. Injection volume: 20 μL. Detector: Refractive Index (Knauer K-2301). Runtime: 26 minutes.

Sample Preparation for GCMS Analysis:

10 μL of supernatants mixed with 5 or 10 μL of 10 mg/mL U-¹³C-Glucose used as internal standard were lyophilized until the samples were completely dried. The resulting material was re-dissolved in 50 μL of acetonitrile. 50 μL of MOX reagent was added to each sample, which were subsequently incubated at 30° C. for 90 minutes. At the end of the incubation period 100 μL of BSTFA was added to each sample. Samples were heated at 50° C. for 30 minutes, cooled to room temperature, transferred into 400 μL glass inserts, and then analyzed by GCMS according to a standard protocol.

### Results

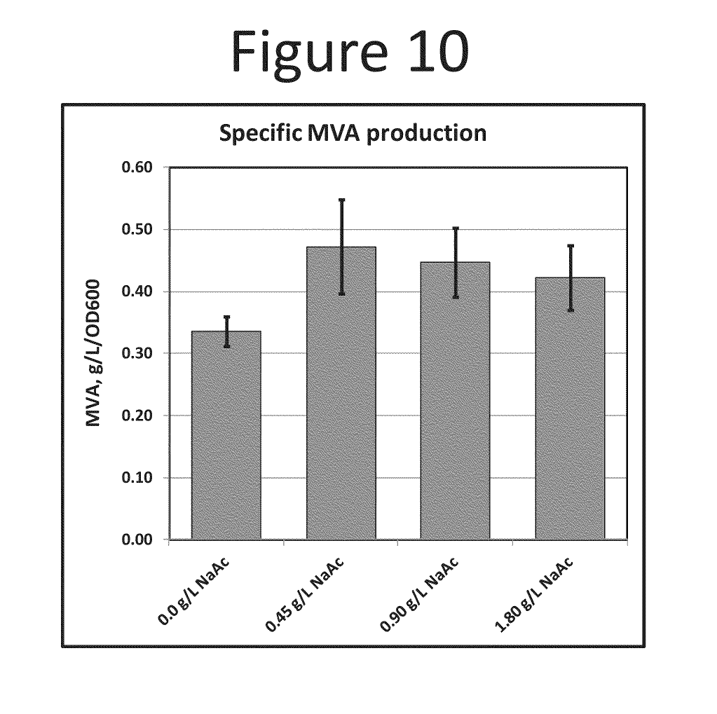

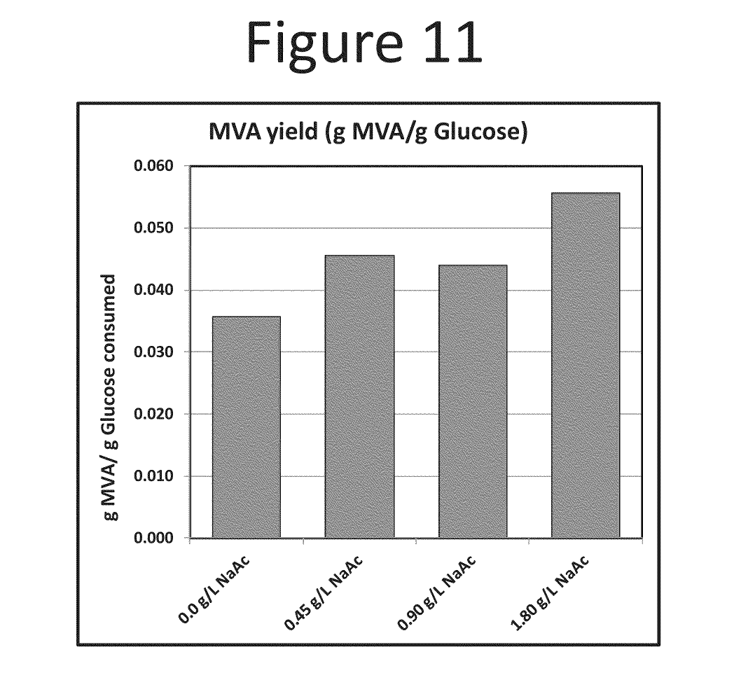

Addition of acetate the cells grown in shake flasks to final concentrations of 0.045 to 0.18% resulted in an increase in specific MVA production (FIG. 10) and MVA yield (FIG. 11) compared to the control with no acetate being added, whereas addition of acetate to a final concentration of 0.045% and 0.090% also resulted in increased MVA titer (0.41±0.07 or 0.38±0.05 g/L MVA, respectively, versus 0.32±0.02 in the control). Lower MVA titer at the end of 3.5 hr incubation period in the presence of 0.18% sodium acetate (0.33±0.04 g MVA/L) is explained by slower cells growth at this concentration of acetate.

These results demonstrate that the efficiency of MVA production from glucose is significantly improved by the addition of acetate to the culture media as shown by the increased yield, titer, and specific productivity in the presence of acetate as compared the control with no addition acetate to the culture media.

### Experiment 5

### Use of Glucose and Acetate to Increase Isoprene Production in 15-L Fermentor Experiment

This experiment was performed to evaluate effects of acetate co-feed on isoprene production from an isoprene producing E. coli strain (DW719) grown in a fed-batch culture at the 15-L scale.

### Materials and Methods

### Medium Recipe (Per Liter Fermentation Medium):

K₂HPO4 7.5 g, MgSO₄*7H₂O 2 g, citric acid monohydrate 2 g, ferric ammonium citrate 0.3 g, yeast extract 0.5 g, 50% sulphuric acid 1.6 mL, 1000× Modified Trace Metal Solution 1 ml. All of the components were added together and dissolved in Di H₂O. This solution was heat sterilized (123° C. for 20 minutes). The pH was adjusted to 7.0 with ammonium hydroxide (28%) and q.s. to volume. Glucose 10 g, Vitamin Solution 8 mL, and antibiotics were added after sterilization and pH adjustment.

### 1000× Modified Trace Metal Solution (Per Liter):

Citric Acids*H2O 40 g, MnSO4*H2O 30 g, NaCl 10 g, FeSO4*7H2O 1 g, CoCl2*6H2O 1 g, ZnSO*7H2O 1 g, CuSO4*5H2O 100 mg, H3BO3 100 mg, NaMoO4*2H2O 100 mg. Each component was dissolved one at a time in Di H2O, pH was adjusted to 3.0 with HCl/NaOH, and then the solution was q.s. to volume and filter sterilized with a 0.22 micron filter.

### Vitamin Solution (Per Liter):

Thiamine hydrochloride 1.0 g, D-(+)-biotin 1.0 g, nicotinic acid 1.0 g, pyridoxine hydrochloride 4.0 g. Each component was dissolved one at a time in Di H2O, pH was adjusted to 3.0 with HCl/NaOH, and then the solution was q.s. to volume and filter sterilized with 0.22 micron filter.

### Macro Salt Solution (Per Liter):

MgSO₄*7H₂O 296 g, citric acid monohydrate 296 g, ferric ammonium citrate 49.6 g. All components were dissolved in water, q.s. to volume and filter sterilized with 0.22 micron filter.

### Feed Solution (Per Kilogram):

Glucose 0.590 kg, Di H₂O 0.393 kg, K2HPO4 7.4 g, and 100% Foamblast882 8.9 g. All components were mixed together and autoclaved. After autoclaving the feed solution, nutrient supplements are added to the feed bottle in a sterile hood. Post sterilization additions to the feed are (per kilogram of feed solution), Macro Salt Solution 5.54 ml, Vitamin Solution 6.55 ml, 1000× Modified Trace Metal Solution 0.82 ml. IPTG solution 21.2 ml of a 10 mg/ml solution (target feed concentration is 100 uM IPTG)

### Construction of Strain DW719:

Strain DW719 (BL21 GI1.2 gltA PL.2 MKKDyI t pgl pgl-, yhfSFRTPyddVIspAyhfS thiFRTtruncIspA, pTrc(IspS variant)_mMVK, pCLPtrcUpper_{—}E.gallinarum) was generated by co-transformation of the host strain CMP1133 (BL21 Δpgl PL.2mKKDyI GI1.2 gltA yhfSFRTPyddVIspAyhfS thiFRTtruncIspA) with a plasmid harboring an isoprene synthase variant and a plasmid carrying upper MVA pathway genes from Enterococcus gallinarum. Following standard molecular biology techniques, the host strain CMP1133 was electroporated with pDW240 (pTrc P.alba IspS MEA-mMVK (Carb50)) and pMCM1225 (pCL-Ptrc-Upper_GcMM_{—}163 (Enterococcus gallinarum EG2). Cells were recovered and plated on selective medium, and individual transformants, resistant to spectinomycin and carbenicillin resulted in strain DW719.

### Experimental Procedure:

This experiment was carried out to monitor isoprene production from glucose at the desired fermentation pH (7.0) and temperature (34° C.). To start each experiment, the appropriate frozen vial of the E. coli (BL21) strain was thawed and inoculated into a flask with tryptone-yeast extract (LB) medium and the appropriate antibiotics. After the inoculum grew to an optical density of approximately 1.0, measured at 550 nm (OD₅₅₀), 500 mL was used to inoculate a 15-L bioreactor and bring the initial tank volume to 5 L.

The batched media had glucose batched in at 9.7 g/L. Induction was achieved by adding isopropyl-beta-D-1-thiogalactopyranoside (IPTG). A shot of IPTG was added to the tank to bring the concentration to 200 uM when the cells were at an OD₅₅₀ of 6. Once the glucose was consumed by the culture, as signaled by a rise in pH, the glucose feed solution was fed to meet metabolic demands at rates less than or equal to 10 g/min. The fermentation was run long enough to determine the maximum cumulative isoprene mass yield on glucose, a total of 60 to 64 hrs elapsed fermentation time.

To test the effect of acetate on isoprene production, acetate was fed to one tank in the form of 20% acetic acid. The acetate was delivered at a rate that approximated 27% (mol/mol) of the Hg produced at 18 hrs, fell to 8% (mol/mol) by 35 hrs, but then was ramped up to 24% (mol/mol) from 35.5 to 39.5 hrs. This feeding profile was used to limit acetate accumulation in the tank. No acetate was fed to the control tank. In both cases, pH in the tanks was controlled by co-feeding ammonium hydroxide.

Isoprene, Oxygen, Nitrogen, and Carbon Dioxide levels in the off-gas were determined independently by two mass spectrometers, an iSCAN (Hamilton Sundstrand), and a Hiden HPR20 (Hiden Analytical) mass spectrometer.

Dissolved Oxygen in the fermentation broth is measured by sanitary, sterilizable probe with an optical sensor provided Hamilton Company.

The glucose and organic acid concentrations in the fermentor broth were determined in broth samples taken at 4 hour intervals by an HPLC analysis using a protocol described in the Experiment 4.

### Results

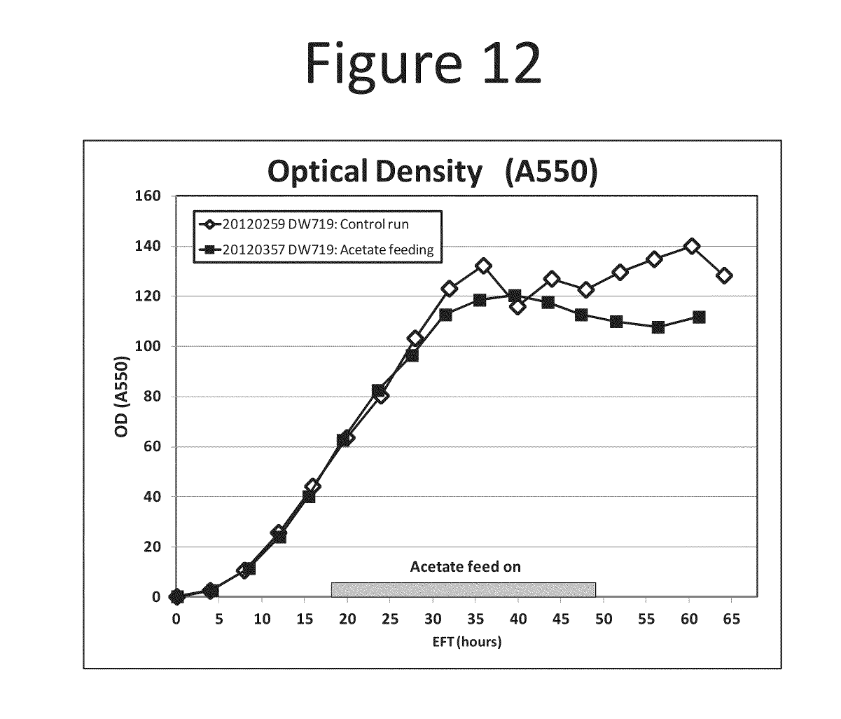

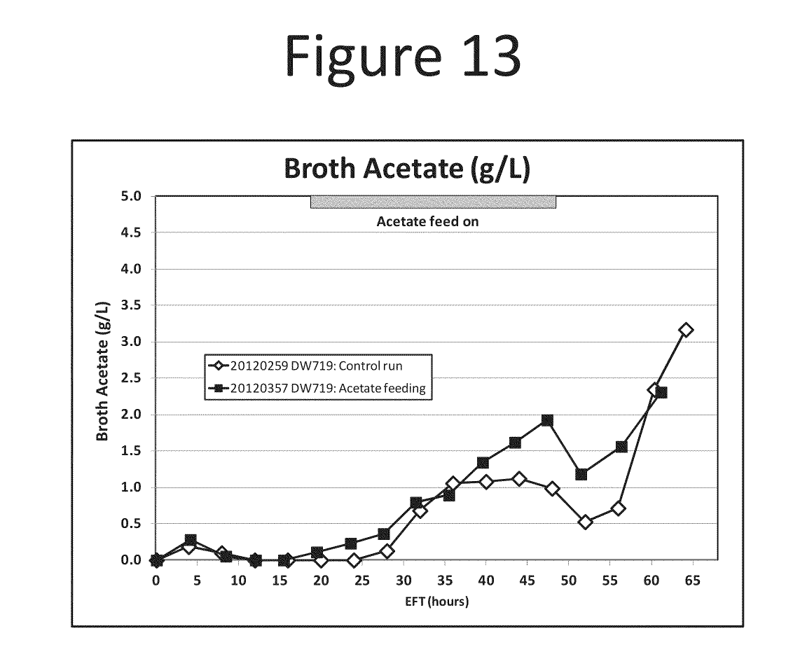

As depicted in FIG. 12, feeding acetate to cells grown in a 15-L fermentor caused a slight decrease in the final OD of the culture compared to the control grown on glucose without acetate co-feed, which is consistent with the acetate effect on cell growth observed in small cultures. Most of the acetate fed to the culture in the 15-L fermentor was metabolized by the cells, as evidenced by very limited accumulation of acetate in the broth (FIG. 13). If acetate wasn't consumed by the cells, the acetate concentration in fermentor broth was expected to reach levels above 10 g/L at 48 hr of the fermentation.

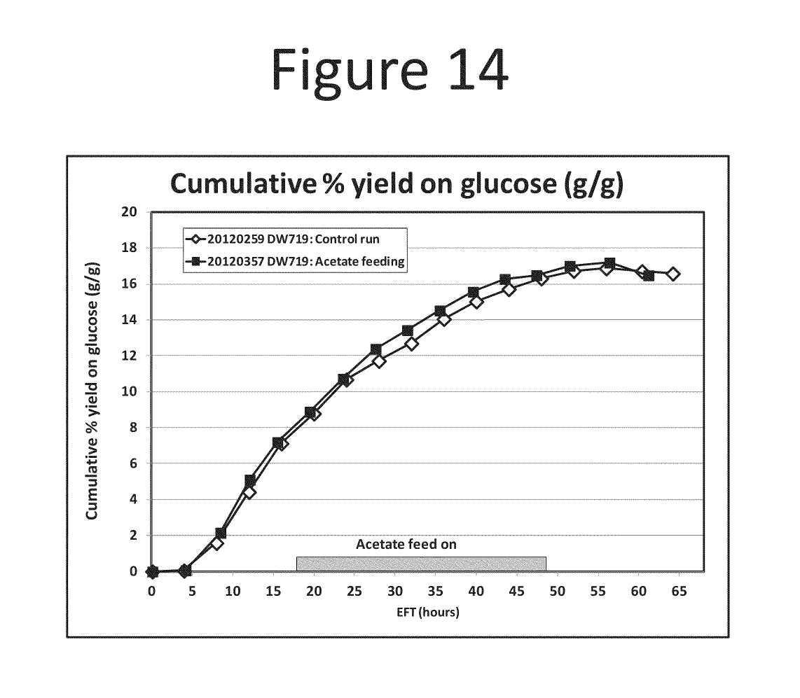

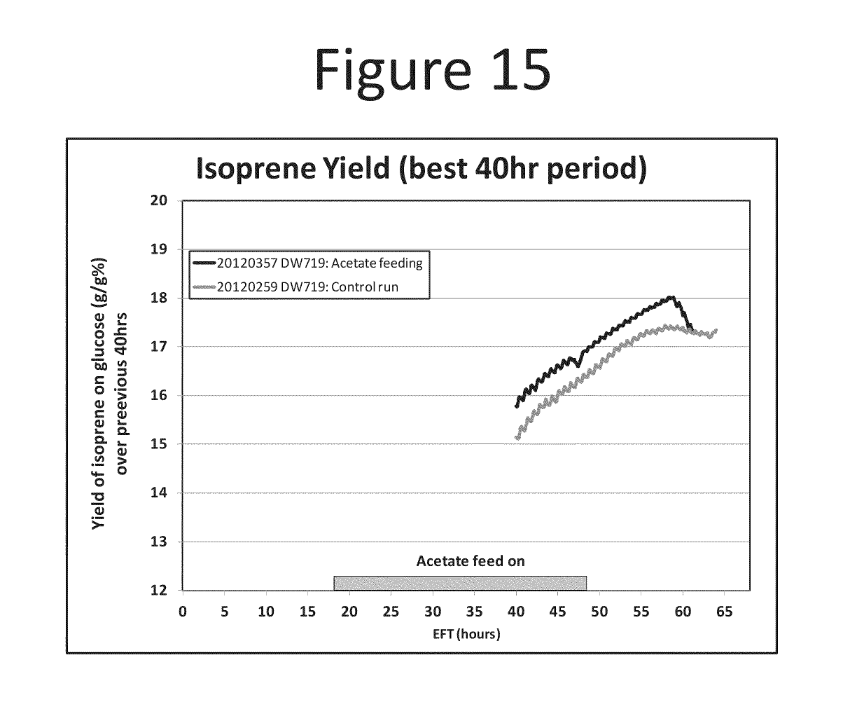

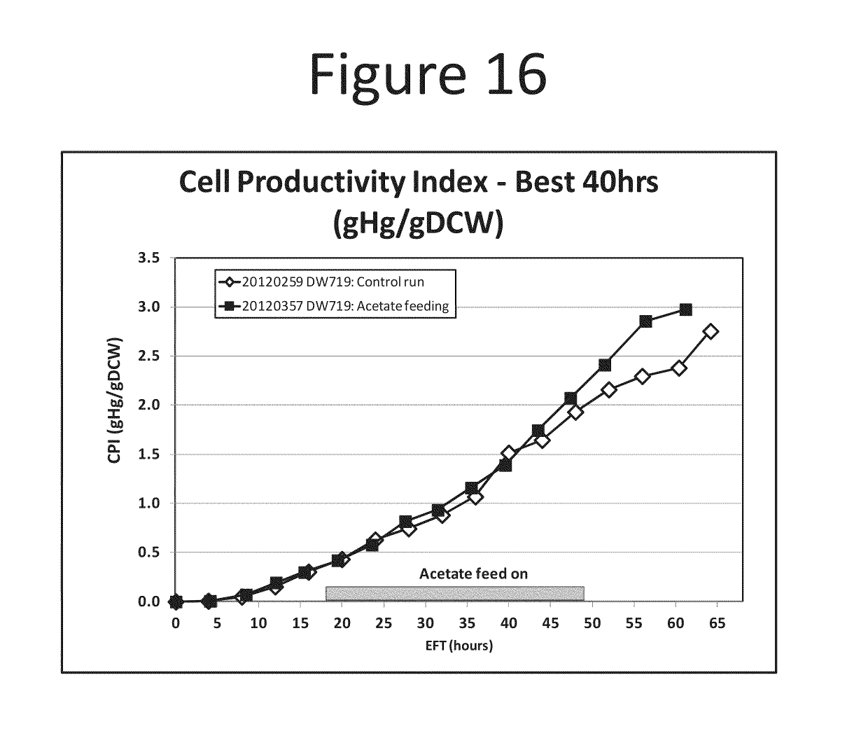

Acetate feeding resulted in increased cumulative yield of isoprene on glucose, which became clearly different from that of the control after about 30 hrs of the fermentation that coincided with accumulation over about 0.5 g/L acetate in the fermentor broth of the acetate-fed culture (FIG. 14). In addition to the increase in cumulative yield of isoprene, acetate feeding improved other metrics of isoprene production, such as the isoprene cumulative yield observed over a 40-hr period (FIG. 15) and the cell productivity index (FIG. 16). Taken altogether, these data demonstrate that co-feeding acetate and glucose noticeably improves the efficiency of the production of isoprene by recombinant host cells in large-scale fermentations.

### Example 6

### The Use of Glucose and Acetate to Increase Amorphadiene or Farnesene Yield in Cultured Microorganisms at 15 L Scale

### Construction of Amorphadiene- or Farnesene Producing Strains

An expression plasmid expressing lad, isoprene synthase and M. mazei mevalonate kinase is modified to replace the gene coding for isoprene synthase by a codon-optimized gene coding for farnesene synthase or amorphadiene synthase. Next, the following expression plasmids are then electroporated (in two steps) into competent E. coli host cells in which farnesyl diphosphate synthase (ispA) is overexpressed (either by altering the promoter and/or rbs on the chromosome, or by expressing it from a plasmid): (i) the plasmid having lad, farnesene synthase or amorphadiene synthase, and M. mazei mevalonate kinase, and (ii) pMCM82 (expression vector MCM82 (see Example 14, U.S. Patent Application Publication No. US201010196977, which is specifically incorporated herein by reference). Colonies are selected on LB+spectinomycin 50 ug/mL+carbenicillin 50 ug/mL+chloramphenicol 25 ug/mL.

### The Use of Glucose and Acetate to Increase Production of Amorphadiene or Farnesene Yield in Cultured Microorganisms at 15 L Scale

### Materials and Methods

### (i) Medium Recipe (Per Liter Fermentation Medium)

K₂HPO4 7.5 g, MgSO₄*7H₂O 2 g, citric acid monohydrate 2 g, ferric ammonium citrate 0.3 g, yeast extract 0.5 g, 50% sulphuric acid 1.6 mL, 1000× Modified Trace Metal Solution 1 ml. All of the components are added together and are dissolved in Di H₂O. This solution is heat sterilized (123° C. for 20 minutes). The pH is adjusted to 7.0 with ammonium hydroxide (28%) and q.s. to volume. Glucose 10 g, Vitamin Solution 8 mL, and antibiotics are added after sterilization and pH adjustment.

### 1000× Modified Trace Metal Solution (Per Liter):

Citric Acids*H2O 40 g, MnSO4*H2O 30 g, NaCl 10 g, FeSO4*7H2O 1 g, CoCl2*6H2O 1 g, ZnSO*7H2O 1 g, CuSO4*5H2O 100 mg, H₃BO3 100 mg, NaMoO4*2H2O 100 mg. Each component is dissolved one at a time in Di H2O, pH is adjusted to 3.0 with HCl/NaOH, and then the solution is q.s. to volume and is filter sterilized with a 0.22 micron filter.

### Vitamin Solution (Per Liter):

Thiamine hydrochloride 1.0 g, D-(+)-biotin 1.0 g, nicotinic acid 1.0 g, pyridoxine hydrochloride 4.0 g. Each component is dissolved one at a time in Di H2O, pH is adjusted to 3.0 with HCl/NaOH, and then the solution is q.s. to volume and is filter sterilized with 0.22 micron filter.

### Macro Salt Solution (Per Liter):

MgSO₄*7H₂O 296 g, citric acid monohydrate 296 g, ferric ammonium citrate 49.6 g. All components are dissolved in water, are q.s. to volume and are filter sterilized with 0.22 micron filter.

### Feed Solution (Per Kilogram):

Glucose 0.590 kg, Di H₂O 0.393 kg, K2HPO4 7.4 g, and 100% Foamblast882 8.9 g. All components are mixed together and are autoclaved. After autoclaving the feed solution, nutrient supplements are added to the feed bottle in a sterile hood. Post sterilization additions to the feed are (per kilogram of feed solution), Macro Salt Solution 5.54 ml, Vitamin Solution 6.55 ml, 1000× Modified Trace Metal Solution 0.82 ml. IPTG solution 21.2 ml of a 10 mg/ml solution (target feed concentration is 100 uM IPTG)

### Experimental Procedure:

This experiment is carried out to monitor amorphadiene- or farnesene production from glucose at the desired fermentation pH (7.0) and temperature (34° C.). To start each experiment, the appropriate frozen vial of the E. coli (BL21) strain is thawed and inoculated into a flask with tryptone-yeast extract (LB) medium and the appropriate antibiotics. Prior to inoculation, an overlay of 20% (v/v) dodecane (Sigma-Aldrich) is added to each culture flask to trap the volatile sesquiterpene product as described previously (Newman et. al., 2006).

Induction is achieved by adding isopropyl-beta-D-1-thiogalactopyranoside (IPTG). A shot of IPTG is added to the tank to bring the concentration to 200 uM when the cells were at an OD₅₅₀ of 6. Once the glucose is consumed by the culture, as signaled by a rise in pH, the glucose feed solution was fed to meet metabolic demands at rates less than or equal to 10 g/min. The fermentation was run long enough to determine the maximum cumulative isoprene mass yield on glucose, a total of 60 to 64 hrs elapsed fermentation time.

To test the effect of acetate on amorphadiene- or farnesene production, acetate is fed to one tank in the form of 20% acetic acid. The acetate is delivered at a rate that approximated 27% (mol/mol) of the amorphadiene- or farnesene produced at 18 hrs, falls to 8% (mol/mol) by 35 hrs, but then is ramped up to 24% (mol/mol) from 35.5 to 39.5 hrs. This feeding profile is used to limit acetate accumulation in the tank. No acetate is fed to the control tank. In both cases, pH in the tanks is controlled by co-feeding ammonium hydroxide.

Samples are taken regularly during the course of the fermentation. At each timepoint, OD600 is measured. Also, amorphadiene or farnesene concentration in the organic layer is assayed by diluting the dodecane overlay into ethyl acetate. Dodecane/ethyl acetate extracts are analyzed by GC-MS methods as previously described (Martin et. al., Nat. Biotechnol. 2003, 21:96-802) by monitoring the molecular ion (204 m/z) and the 189 m/z fragment ion for amorphadiene or the molecular ion (204 m/z) for farnesene. Amorphadiene or farnesene samples of known concentration are injected to produce standard curves for amorphadiene or farnesene, respectively. The amount of amorphadiene or farnesene in samples is calculated using the amorphadiene or farnesene standard curves, respectively.

### (iii) Results

The amorphadiene or farnesene strains cultured in the presence of acetate are compared to the same background without acetate co-feed, to determine the specific productivity, yield, CPI and/or titer of amorphadiene or farnesene. It is expected that the amorphadiene or farnesene strains cultured in the presence of acetate display improved efficiency in the production of amorphadiene or farnesene as compare to the strains cultured in the absence of acetate.

### (iv) References

Newman, J. D., Marshal, J. L., Chang, M. C. Y., Nowroozi, F., Paradise, E. M., Pitera, D. J., Newman, K. L., Keasling, J. D., 2006. High-level production of amorpha-4,11-diene in a two-phase partitioning bioreactor of metabolically engineered E. coli. Biotechnol. Bioeng. 95:684-691.
Martin, V. J., Pitera, D. J., Withers, S. T., Newman, J. D., Keasling, J. D., 2003. Engineering a mevalonate pathway in E. coli for production of terpenoids. Nat. Biotechnol. 21:796-802.

The examples, which are intended to be purely exemplary of the invention and should therefore not be considered to limit the invention in any way, also describe and detail aspects and aspects of the invention discussed above. The foregoing examples and detailed description are offered by way of illustration and not by way of limitation. All publications, patent applications, and patents cited in this specification are herein incorporated by reference as if each individual publication, patent application, or patent were specifically and individually indicated to be incorporated by reference. In particular, all publications cited herein are expressly incorporated herein by reference for the purpose of describing and disclosing compositions and methodologies which might be used in connection with the invention. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A method for improving the efficiency of the production of isoprene by recombinant host cells in culture, the method comprising culturing said recombinant host cells in culture media comprising a carbon source and acetate under suitable conditions for the production of isoprene,
wherein said recombinant host cells comprise one or more heterologous nucleic acids encoding an isoprene synthase polypeptide;
wherein said recombinant host cells are capable of producing isoprene; and
wherein isoprene production by said recombinant host cells cultured in the culture media comprising a carbon source and acetate is improved compared to the isoprene production by said recombinant host cells cultured in culture media comprising a carbon source in the absence of acetate.

2. The method of claim 1, wherein said improved production of isoprene is characterized by an increase in: (i) the specific productivity, (ii) the cumulative yield, (iii) the cumulative yield, or (iv) Cell Productivity Index.

3. The method of claim 2, wherein said improved production of isoprene is characterized by an increase in the specific productivity.

4. The method of claim 2, wherein said improved production of isoprene is characterized by an increase in the cumulative yield.

5. The method of claim 2, wherein said improved production of isoprene is characterized by an increase in the cumulative yield.

6. The method of claim 2, wherein said improved production of isoprene is characterized by an increase in the Cell Productivity Index.

7. The method of claim 1, wherein the isoprene synthase polypeptide is a plant isoprene synthase polypeptide.

8. The method of claim 7, wherein the isoprene synthase polypeptide is a polypeptide from Pueraria or Populus or a hybrid, Populus alba×Populus tremula.

9. The method of claim 8, wherein the isoprene synthase polypeptide is selected from the group consisting of Pueraria montana or Pueraria lobata, Populus tremuloides, Populus alba, Populus nigra, and Populus trichocarpa.

10. The method of claim 7, wherein the plant isoprene synthase polypeptide is a kudzu isoprene synthase polypeptide.

11. The method of claim 1, wherein the cells further comprise one or more heterologous nucleic acid encoding one or more MVA pathway polypeptides.

12. The method of claim 11, wherein the cells further comprise one or more heterologous nucleic acid encoding the entire MVA pathway.

13. The method of claim 1, wherein the cells further comprise a heterologous nucleic acid encoding an isopentyl-diphosphate isomerase (IDI) polypeptide.

14. The method of claim 1, wherein the cells further comprise a heterologous nucleic acid encoding a DXS polypeptide.

15. The method of claim 1, wherein the recombinant host cells are gram-positive bacterial cells, gram-negative bacterial cells, fungal cells, filamentous fungal cells, algal cells or yeast cells.

16. The method of claim 1, wherein the recombinant host cells are selected from the group consisting of Bacillus subtilis, Streptomyces lividans, Streptomyces coelicolor, Streptomyces griseus, Escherichia coli, Pantoea citrea, Trichoderma reesei, Aspergillus oryzae and Aspergillus niger, Saccharomyces cerevisieae and Yarrowia lipolytica.

17. The method of any of claim 1, wherein the concentration of acetate is at least about 0.01% to about 1.5%.

18. A method for improving the efficiency of the production of isoprenoid precursors by a recombinant host cell, the method comprising:
a. providing one or more recombinant host cells comprising one or more heterologous nucleic acids encoding one or more MVA pathway polypeptides; and
b. culturing said recombinant host cells in the presence of glucose and acetate under suitable conditions for the production of isoprenoid precursors;
wherein the production of isoprenoid precursors by said recombinant host cells cultured in the presence of glucose and acetate is improved compared to the production of isoprenoid precursors by said recombinant host cells cultured in the presence of glucose alone.

19. The method of claim 18, wherein said improved production of an isoprenoid precursor is characterized by an increase in: (i) the specific productivity, (ii) yield, or (iii) titer.

20. The method of claim 18, wherein the isoprenoid precursor is selected from group consisting of mevalonate (MVA), DMAPP or IPP.

21. The method of claim 18, wherein the isoprenoid precursor is mevalonate (MVA).

22. The method of claim 18, wherein the one or more heterologous nucleic acids is placed under an inducible promoter or a constitutive promoter.

23. The method of claim 18, wherein the one or more heterologous nucleic acids is cloned into a multicopy plasmid.

24. The method of claim 18, wherein the cells are selected from the group consisting of bacterial cells, fungal cells, or algal cells.

25. A method for improving the efficiency of the production of isopreniods by recombinant host cells in culture, the method comprising culturing said recombinant host cells in culture media comprising a carbon source and acetate under suitable conditions for the production of isopreniods,
wherein said recombinant host cells comprise (i) one or more heterologous nucleic acid encoding the entire MVA pathway and (ii) one or more heterologous nucleic acids encoding for a polyprenyl pyrophosphate synthase;
wherein said recombinant host cells are capable of producing an isoprenoid; and
wherein isoprenoid production by said recombinant host cells cultured in the presence of glucose and acetate is improved compared to the isoprenoid production by said recombinant host cells cultured in the presence of glucose alone.

26. The method of claim 25, wherein said improved production of isoprene is characterized by an increase in: (i) the specific productivity, (ii) the cumulative yield, (iii) the cumulative yield, or (iv) Cell Productivity Index.

27. The method of any one of claim 25, wherein the isoprenoid is selected from group consisting of monoterpenes, diterpenes, triterpenes, tetraterpenes, sequiterpene, and polyterpene.

28. The method of claim 27, wherein the isoprenoid is a sesquiterpene.

29. The method of any one of claim 27, wherein the isoprenoid is selected from the group consisting of abietadiene, amorphadiene, carene, α-farnesene, β-farnesene, farnesol, geraniol, geranylgeraniol, linalool, limonene, myrcene, nerolidol, ocimene, patchoulol, β-pinene, sabinene, γ-terpinene, terpindene and valencene.

30. The method of claim 25, wherein the one or more heterologous nucleic acids is placed under an inducible promoter or a constitutive promoter.

31. The method of claim 25, wherein the one or more heterologous nucleic acids is cloned into a multicopy plasmid.

32. The method of claim 25, wherein the cells are selected from the group consisting of bacterial cells, fungal cells, or algal cells.

33. The method of claim 25, wherein the concentration of acetate is at least about 0.01% to about 1.5%.
